SEQUENCE LISTING (2 sequences)

SEQ ID NO: 1 - DNA, 4422 bp, Homo Sapiens Aromatase Polynucleotide
gggagtttctggagggctgaacacgtggaggcaaacaggaaggtgaagaagaacttatcctatcaggacggaaggtcctgtgctcgggatcttccagacgtcgcgactctaaattgccccctctgaggtcaaggaacacaagatggttttggaaatgctgaacccgatacattataacatcaccagcatcgtgcctgaagccatgcctgctgccaccatgccagtcctgctcctcactggcctttttctcttggtgtggaattatgagggcacatcctcaataccaggtcctggctactgcatgggaattggacccctcatctcccacggcagattcctgtggatggggatcggcagtgcctgcaactactacaaccgggtatatggagaattcatgcgagtctggatctctggagaggaaacactcattatcagcaagtcctcaagtatgttccacataatgaagcacaatcattacagctctcgattcggcagcaaacttgggctgcagtgcatcggtatgcatgagaaaggcatcatatttaacaacaatccagagctctggaaaacaactcgacccttctttatgaaagctctgtcaggccccggccttgttcgtatggtcacagtctgtgctgaatccctcaaaacacatctggacaggttggaggaggtgaccaatgaatcgggctatgtggacgtgttgacccttctgcgtcgtgtcatgctggacacctctaacacgctcttcttgaggatccctttggacgaaagtgctatcgtggttaaaatccaaggttattttgatgcatggcaagctctcctcatcaaaccagacatcttctttaagatttcttggctatacaaaaagtatgagaagtctgtcaaggatttgaaagatgccatagaagttctgatagcagaaaaaagacgcaggatttccacagaagagaaactggaagaatgtatggactttgccactgagttgattttagcagagaaacgtggtgacctgacaagagagaatgtgaaccagtgcatattggaaatgctgatcgcagctcctgacaccatgtctgtctctttgttcttcatgctatttctcattgcaaagcaccctaatgttgaagaggcaataataaaggaaatccagactgttattggtgagagagacataaagattgatgatatacaaaaattaaaagtgatggaaaacttcatttatgagagcatgcggtaccagcctgtcgtggacttggtcatgcgcaaagccttagaagatgatgtaatcgatggctacccagtgaaaaaggggacaaacattatcctgaatattggaaggatgcacagactcgagtttttccccaaacccaatgaatttactcttgaaaattttgcaaagaatgttccttataggtactttcagccatttggctttgggccccgtggctgtgcaggaaagtacatcgccatggtgatgatgaaagccatcctcgttacacttctgagacgattccacgtgaagacattgcaaggacagtgtgttgagagcatacagaagatacacgacttgtccttgcacccagatgagactaaaaacatgctggaaatgatctttaccccaagaaactcagacaggtgtctggaacactagagaaggctggtcagtacccactctggagcatttctcatcagtagttcacatacaaatcatccatccttgccaatagtgtcatcctcacagtgaacactcagtggcccatggcattttataggcatacctcctatgggttgtcaccaagctaggtgctatttgtcatctgctcctgttcacaccagagaaccaggctacaagagaaaaagcagaggccaagagtttgagggagaaatagtcggtgaagaaaccgtatccataaagacccgattccaccaaatgtgctttgagaaggataggccttcattaacaaaatgtatgtctggttccccagtagagctctactgcctcaacccaaggggatttttatgtctggggcagaaacactcaagttgattagaaagaccaggccaatgtcagggtacctggggccaaacccacctgctagtgtgaattaaagtactttaattttgttttctgtggaggtggaaaagcaacattcatagtctttggagaaatgcttagaaattcagcatttgacccttgctgtgaattaagcccaattaattcctgtttgtctacatatgatctgtctgtggcaaaagtttaatcagaggaaattctttcccagtctgtcgatttatgcctcagccacttgcctgtgctacaattcattgtgttacctgtagattcaggtaatacaaactatatataatcatcaagtaatacaaactaatttagtaatagcctgggttaagtattattagggccctgtgtctgctgtagaaaaaaaaattcacatgatgcacttcaaattcaaataaaaatccttttggcatgttcccatttttgcttagctcaattagtgtggctaaccaagagataactgtaaatgtgacattgatttgctcttactacagcttcagtgattgggggaggaaaagtcccaacccaatgggctcaaacttctaaggggtactcctctcatccccttatccttctccctcgacattttctccctctttcttcccatgaccccaaagccaagggcaacagatcagtaaagaacgtggtcagagtagaacccctgaagtattttttaatcctacctcaaaatttaacagttacctgagagatttaacattatctagttcattgaatcattgtatgtggtcatggataaattgcacaccttggaattcgctttctaaaggaaatcaaatgaatggaggaactttccaaacaccactttacttgtgttatatagccaatataactatctctactgaatgtcattgaaaaactaaaaaattaaacttatttacaaataggtaaatatttgtcattgaatccattgccatcccatttgactgttcttttcatcctactgtctagtaataagctgagtataagatgacagtgtaatctccctgaaagcaggagctactttctttcttttgtaatctatttccatccccatttccctgtcctgtctccctgtattcactcccaagctcagttctgaatagacattcctgctcagagatactcccaactgatgcagaaaccaaataaagaggtaggtattccaagaattcaagaatggacattagtaaagaataaaacatttatttgagcttggaattatttggatcatctatatggcctaaaaatatatggactatgcctgtgtacctgaatacgtatgtagtcaggtcaagacaatcatccaaataacttagacccctaaaagcaaggccaggatttgcaatttaatgtgtcccaattaattcacttgaaaattagtaacactctgtttacgttgcctctggctggagctgcatggtggaagaagcccaactttggatccatgtacttcacccatccaatactcttgggacatttatgtgtattttatctgtatatatgaagccaatgtctatgtctacacagtcaaagtgaaatgcatgtttgatatagctgtacatagatatctattttgcaggtacaaaaatatcctgggggaaaactgggagtggaagggtggggggtgggagtgagggacatgggggagggacaggaagaggagaagtgttggtttgaacgatccaagcaaactctcccagaatcaaattacctgggtagttgttcaacttttcactctgcttagcctgtatagacaaaccccatatatttgtagaggcttggccttggaattctggaataccattggcttttcagtaggctgatgaacacattttgaaaattctattatcttcagaattttgccccattgttaagtgcttaaccgtcactcttgaatgtgcaatgtgctgtggattccattttcatcagttctgaaagaactgcaatgtgtaaattatcagtgaaatgcatgcatataagggctctatcattatcaaattgtaaggacaattgtacccttctatatctttgggcatgctagacacccccatgccttcattgagatcccattttccccctctcaagtggaaaataatcacatccagcaagctctctcattattgagaaataccatttggaaattgccactttttattcctaagcagcacctttcactgttcatgatgctaatgttccacaaaagcatgtgccattggcccactgaaggatagagggacccttttcaatctatatcagctgggctctgggactgaatctctcacctattcttgcagaaagacatactaattaaaccttgtcaaagtaaaaaaaaaaaaaaaaaa

SEQ ID NO: 2 - protein, 503 aa, Homo Sapiens Aromatase Polypeptide
MVLEMLNPIHYNITSIVPEAMPAATMPVLLLTGLFLLVWNYEGTSSIPGPGYCMGIGPLISHGRFLWMGIGSACNYYNRVYGEFMRVWISGEETLIISKSSSMFHIMKHNHYSSRFGSKLGLQCIGMHEKGIIFNNNPELWKTTRPFFMKALSGPGLVRMVTVCAESLKTHLDRLEEVTNESGYVDVLTLLRRVMLDTSNTLFLRIPLDESAIVVKIQGYFDAWQALLIKPDIFFKISWLYKKYEKSVKDLKDAIEVLIAEKRRRISTEEKLEECMDFATELILAEKRGDLTRENVNQCILEMLIAAPDTMSVSLFFMLFLIAKHPNVEEAIIKEIQTVIGERDIKIDDIQKLKVMENFIYESMRYQPVVDLVMRKALEDDVIDGYPVKKGTNIILNIGRMHRLEFFPKPNEFTLENFAKNVPYRYFQPFGFGPRGCAGKYIAMVMMKAILVTLLRRFHVKTLQGQCVESIQKIHDLSLHPDETKNMLEMIFTPRNSDRCLEH

(19) United States
(12) Patent Application Publication
(10) Pub. No.: US 2010/0331203 A1
(43) Pub. Date: Dec. 30, 2010

(54) AROMATASE EXPRESSION PREDICTS SURVIVAL IN WOMEN WITH NON-SMALL CELL LUNG CANCER

(75) Inventors: Lee Goodglick, Los Angeles, CA (US); Richard J. Pietras, Sherman Oaks, CA (US); Vei Mah, Davis, CA (US); David B. Seligson, Los Angeles, CA (US); David S. Chia, Santa Monica, CA (US); Diana C. Marquez-Garban, Los Angeles, CA (US); Michael C. Fishbein, Encino, CA (US)

Correspondence Address: GATES & COOPER LLP;HOWARD HUGHES CENTER, 6701 CENTER DRIVE WEST, SUITE 1050, LOS ANGELES, CA, 90045, US

(73) Assignee: THE REGENTS OF THE UNIVERSITY OF CALIFORNIA, Oakland, CA (US)

(21) Appl. No.: 12/681,579

(22) PCT Filed: Oct. 6, 2008
(86) PCT No.: PCT/US08/78992

§ 371 Date: Apr. 2, 2010

Related U.S. Application Data
(60) Provisional application No. 60/997,867, filed on Oct. 5, 2007.

(51) Int. Cl. C40B 30/04; C12Q 1/26; G01N 33/573; C12Q 1/68

(52) U.S. Cl. 506/9; 435/25; 435/7.4; 435/6

(57) ABSTRACT

The present invention relates to the discovery that the level of aromatase polypeptide expression in non-small cell lung carcinomas can be used for example to provide information useful in prognostic and therapeutic methodologies in women having or suspected of having this cancer.

### REFERENCE TO RELATED APPLICATIONS

This application claims priority under Section 119(e) from U.S. Provisional Application Ser. No. 60/997,867 filed Oct. 5, 2007, the contents of which are incorporated herein by reference.

### STATEMENT OF GOVERNMENT SUPPORT

This invention was made with United States Government support under National Institutes of Health Grants CA086366 and CA090388. The United States Government may have certain rights in the invention.

### FIELD OF THE INVENTION

The invention described herein relates to prognostic and therapeutic methodologies based on observations of the expression of the aromatase gene.

### BACKGROUND OF THE INVENTION

Cancers are the second most prevalent cause of death in the United States, causing 450,000 deaths per year. One in three Americans will develop cancer, and one in five will die of cancer. While substantial progress has been made in identifying some of the environmental and hereditary factors that contribute to the development of a number of cancers, there continues to be a need for substantial improvement in the diagnosis and therapy of cancers including cancers of the lung.

Lung cancer remains the leading cause of cancer-related deaths for both men and women (see, e.g. Jemal et al., CA Cancer J Clin 2007; 57:43-66). In the United States alone, an estimated 213,380 new cases of lung cancer will be diagnosed in 2007 and approximately 160,390 individuals will die due to this disease. Very little is understood about the underlying mechanisms of development and progression of these neoplasms. Moreover, the search for effective therapeutic modalities continues to be elusive. Interestingly, the incidence of lung cancer in men has somewhat declined over the last decade while the reported cases in women has steadily increased. A primary factor for this is the increased level of smoking by women; however, this does not appear to be the only determinant as non-smokers who develop lung cancer are predominantly women. Recent evidence suggests that estrogen may play a role in normal lung function as well as the development of lung cancer (see, e.g. Stabile et al., Cancer Res 2002; 62:2141-50; Stabile et al., Cancer Res 2005; 65:1459-70; Pietras et al., Steroids 2005; 70:372-81; Hershberger et al., Cancer Res 2005; 65:1598-605; Weinberg et al., Cancer Res 2005; 65:11287-91; Patrone et al., Mol Cell Biol 2003; 23:8542-52; Kononen et al., Nat Med 1998; 4:844-7; Mollerup et al., Lung Cancer 2002; 37:153-9; Omoto et al., Biochem Biophys Res Commun 2001; 285:340-7; and Taioli et al., J Natl. Cancer Inst 1994; 86:869-70). Estrogen receptors (ER) α and β are expressed in normal lung and many non-small cell lung carcinomas (NSCLC). In both in vitro and in vivo models, 17β-estradiol is mitogenic for NSCLC cells and stimulates gene transcription (see, e.g. Stabile et al., Cancer Res 2002; 62:2141-50; Stabile et al., Cancer Res 2005; 65:1459-70; Hershberger et al., Cancer Res 2005; 65:1598-605). These effects are inhibited by the ER antagonist, Faslodex (fulvestrant), as is tumor formation in a human NSCLC xenograph model in vivo (see, e.g. Stabile et al., Cancer Res 2002; 62:2141-50).

A key enzyme in estrogen biosynthesis is aromatase (CYP19) (see, e.g. Bulun et al., J Steroid Biochem Mol Biol 2003; 86:219-24; Brodie et al., Semin Oncol 2003; 30:12-22; Kinoshita et al., Cancer Res 2003; 63:3546-55). Aromatase, a member of the cytochrome P450 family, converts the androgens androstenedione and testosterone to estrone and estradiol, respectively. In addition to its expression in the ovary and placenta, aromatase is present in male and female extra-gonadal tissues including breast, lung, brain, and liver (see, e.g. Simpson et al., Endocr Rev 1994; 15:342-55). Aromatase expression is elevated in certain malignancies, such as breast and endometrial carcinomas, prompting the theory that the tumor-promoting properties caused by stimulation of estrogen receptor pathways could be enhanced by circulating estrogen as well as by localized autocrine or paracrine production of the steroid hormone. This rationale led to several clinical studies that confirmed the antitumor efficacy of aromatase inhibitors in breast cancer (see, e.g. Brodie et al., Semin Oncol 2003; 30:12-22; Smith et al., N Engl J Med 2003; 48:2431-42; Coombes et al., N Engl J Med 2004; 350:1081-92; Gould et al., Curr Opin Obstet Gynecol 2006; 18:41-6).

While the identification aromatase as an effector of oncogenic processes in breast cancer facilitates efforts to diagnose and treat this cancer, there is need for an analysis of the role of this polypeptide in other malignancies. The disclosure below provides studies of the expression levels of aromatase in non-small cell lung carcinomas showing that observation of the level of aromatase polypeptide expressed by a NSCLC can used to obtain diagnostic and prognostic information on this malignancy.

### SUMMARY OF THE INVENTION

Estrogen signaling is critical in the progression of tumors that bear estrogen receptors. In most patients with breast cancer, inhibitors that block interactions of estrogen with its receptors or suppress the production of endogenous estrogens, are important interventions in the clinic. Recent evidence now suggests that estrogen contributes to the pathogenesis of non-small cell lung cancer (NSCLC). In this context, the disclosure provided below shows that aromatase, the enzyme that converts androgens to estrogens, is expressed in these lung malignancies and can be used as a strong predictor of clinical outcome. Briefly, the disclosure provided herein shows the results of an examination of the level of protein expression of aromatase in 422 non-small cell lung cancer patients using a high-density tissue microarray. Significantly, this analysis shows that lower levels of aromatase predicted a greater chance of survival in women 65 years and older. Within this population, the prognostic value of aromatase was greatest in early, stage I/II lung cancer. In addition, for women with no history of smoking, lower aromatase levels are a strong predictor of survival. These findings demonstrate that aromatase is an early stage predictor of survival in some women with NSCLC and that aromatase expression levels can be used as an early stage prognostic indictor for survival versus death for women 65 years and older. Moreover, these findings further provide evidence that lower aromatase expression levels predict a greater probability of survival in women who have NSCLC with no history of smoking and further that women whose lungs cancers have higher aromatase levels will benefit from aromatase inhibitor therapy.

Embodiments of the invention are based on the observation that, in certain individuals, the level of aromatase polypeptide expressed by a NSCLC cell can be correlated with factors such as survival. In this context, a typical embodiment of the invention is a method of examining a human non-small cell lung carcinoma obtained from a patient comprising the steps of observing the level of aromatase (SEQ ID NO: 2) polypeptide expressed in the non-small cell lung carcinoma, wherein the patient is female and is at least 65 years old. Certain embodiments of the invention further comprise the step of using the observed level of aromatase (SEQ ID NO: 2) polypeptide expressed in the non-small cell lung carcinoma to determine a prognosis for the patient. In typical embodiments of the invention, the prognosis comprises an estimate of the probability of the patient surviving for at least 6 months, 1, 2, 3, 4, 5, 6, 7, 8, 9 or 10 years from the time of the examination. Other embodiments of the invention comprise the step of using the observed level of aromatase (SEQ ID NO: 2) polypeptide expressed by the non-small cell lung carcinoma to evaluate a course of therapy for the patient. In certain embodiments of the invention, a high level of aromatase expression identifies the patient as a candidate for treatment with an aromatase inhibitor. Optionally in such embodiments, a course of therapy for a patient exhibiting such an aromatase profile includes administering one or more aromatase inhibitors to the patient.

A number of factors can be considered in the diagnostic and prognostic methods of the invention. For example, in addition to aromatase expression, factors such as age and gender, and a person's lifestyle behavior can be considered. In one such embodiment of the invention, a methodological step includes determining the patient's age and if the patient has a lifetime history of smoking more than 100 cigarettes. In addition, in certain embodiments of the invention, the methods which examine aromatase expression are combined with other methods commonly used in cancer diagnoses and prognosis. For example in one such embodiment of the invention, a tissue sample (e.g. from a biopsy) is examined to determine the stage or grade of cancer cells in the same (if any). In typical embodiments of the invention, one examines a sample having or suspected of containing non-small cell lung carcinoma cells to observe the presence (or absence) of a stage I, stage II, stage III or stage IV non-small cell lung carcinoma.

A number of methods for examining aromatase expression can be used in embodiments of the invention. In some embodiments of the invention, the level of aromatase polypeptides expressed by a non-small cell lung carcinoma are observed using an assay of aromatase polypeptide enzymatic activity. Alternatively, a level of aromatase polypeptide expressed by the non-small cell lung carcinoma is observed using an antibody that binds aromatase polypeptide. In one such embodiment of the invention, the antibody is used in an immunohistochemical assay of cytoplasmic aromatase staining intensity; the assay comprises a numerical scale of staining intensity values including a mean or median staining intensity value; a staining intensity value above the mean or median staining intensity value characterizes the cell as having a high level of aromatase expression; and a staining intensity value below the mean or median staining intensity value characterizes the cell as having a low level of aromatase expression. In some embodiments of the invention, the level of aromatase polypeptide expressed by the non-small cell lung carcinoma is observed using a high-density tissue microarray. Optionally in the methods of the invention, the staining intensity value is quantified using image analysis software.

In other embodiments of the invention, the level of aromatase polypeptide expressed in the non-small cell lung carcinoma is observed indirectly by using a polymerase chain reaction to observe the level of aromatase mRNA in the cell that encode the aromatase polypeptide; and correlating the level of aromatase mRNA observed in the cell with the level of aromatase polypeptide expressed in the cell. In one such embodiment of the invention, the level of aromatase polypeptide expressed in the non-small cell lung carcinoma is observed by: observing the level of aromatase mRNA in the cell that encode the aromatase polypeptide; and correlating the level of aromatase mRNA observed in the cell with the level of aromatase polypeptide expressed in the cell. Optionally in such methods, the aromatase mRNA is observed in the cell using a polynucleotide primer or probe that hybridizes to the aromatase mRNA. Such methods can be combined with further methodological steps, for example, the step of determining if a cell in the tissue sample is a stage I/II non-small cell lung carcinoma. In typical embodiments of the invention, such steps are used to obtain information useful for determining a prognosis or therapy for a patient of a selected gender and/or age category and/or specific behavioral history, by for example, determining if the patient has a lifetime history of smoking more than 100 cigarettes; and/or determining the age of the patient and/or determining the gender of the patient.

Another illustrative embodiment of the invention is a method of obtaining information useful for determining a prognosis or therapy for a female patient having or suspected of having a human non-small cell lung carcinoma, the method comprising observing a level of aromatase (SEQ ID NO: 2) polypeptide expressed in a tissue sample obtained from the patient that includes or is suspected of including the human non-small cell lung carcinoma cells; and then correlating the level of aromatase polypeptide observed in the cells with a prognosis or therapy for non-small cell lung carcinoma that is associated with the observed level of aromatase (SEQ ID NO: 2) polypeptide expressed by the cells. In certain embodiments of the invention, the level of aromatase polypeptide expressed in the tissue sample is determined using an immunohistochemical staining technique; and the method further comprises determining the percentage of cells in the tissue sample that are stained in the immunohistochemical staining technique. Optionally in such methods, the level of aromatase polypeptide expressed in the tissue sample is determined using an immunohistochemical staining technique; the method is preformed on a plurality of patients; and the plurality of patients are stratified into those having a staining intensity in the immunohistochemical staining technique that is either above or below a mean or median staining intensity value on a numerical scale of staining intensity values derived from a plurality of stained cells having a plurality of levels of aromatase expression. Optionally in these methods, the stratification is used to predict the probability of the patient surviving for at least 1, 2, 3, 4 or 5 years from the time of the examination; and/or to identify patients likely to respond to a therapeutic regimen comprising an aromatase inhibitor.

Other embodiments of the invention include kits for characterizing a mammalian NSCLC tumor or cell, the kit comprising: an antibody that binds aromatase (SEQ ID NO: 1); and/or a polynucleotide primer or probe that hybridizes to the aromatase gene or transcript (SEQ ID NO: 2). Typically the kit further comprises instructions for use of the enclosed materials as well as reagents commonly used to observe proteins and nucleic acids such as a secondary antibodies which bind to one of a primary antibody, buffers, reagents and the like.

### BRIEF DESCRIPTION OF THE FIGURES

FIG. 1. Growth-promoting effects of estrogen and aromatase substrate on non-small cell lung tumor xenografts in vivo. A549 lung tumor cells which express endogenous aromatase were implanted as subcutaneous xenografts in ovariectomized female athymic mice. Mice were then divided into 3 groups (5 animals per group). ▪ Animals were given daily subcutaneous treatments of the aromatase substrate androstenedione as before (see, e.g. Weinberg et al., Cancer Res 2005; 65:11287-91). Animals were given daily subcutaneous treatments of estradiol in a biodegradable binder as described (see, e.g. Pietras et al., Oncogene 1995; 10:2435-46). ▴ Control animals were supplemented with subcutaneous injection of vehicle alone. Growth of A549 tumors was significantly increased in mice treated with androstenedione and estradiol as compared to control animals (P<0.001 for both).

FIG. 2. Aromatase expression in lung tissue samples. (a-g) Immunohistochemical staining of aromatase. Expression of aromatase in the cytoplasm of representative cases of lung adenocarcinomas showing weak (a) and strong (b) staining. Expression of aromatase in the cytoplasm of representative cases of lung squamous cell carcinomas showing weak (c) and strong (d) staining. Aromatase expression in histologically normal alveolar lining (e) and bronchial epithelial cells with low (f) and high (g) expression. In all experiments, substitution of anti-aromatase Ab with species-matched non-immune Ab gave no staining. (h) Aromatase expression distribution as classified by TMA spot-level histological category. Shown here is the mean integrated value of aromatase expression+SEM. Only small cell carcinoma showed significant differences from other categories (P<0.001 compared to large cell carcinoma, adenocarcinoma, and squamous cell carcinoma; P=0.005 compared to bronchial epithelial cells).

FIG. 3. Levels of NSCLC aromatase protein expression correlate well with tumor enzyme activity. Frozen archival non-small cell lung cancer specimens from 7 males and 5 females were examined for aromatase enzyme activity and for protein expression. These specimens correlated to cases on the TMA. Enzyme activity was determined by radioassay and protein expression was assessed by IHC as described in Materials and Methods. The correlation between IHC and aromatase activity was then assessed by linear regression analysis with the correlation coefficient r=0.95.

FIGS. 4a-d. Aromatase expression levels predict probability of survival in women with NSCLC. All panels show Kaplan-Meier survival plots for individuals with NSCLC. Solid lines show lower aromatase expression (<1.5 mean integrated intensity) and dashed lines show higher aromatase expression (>1.5 mean integrated intensity). N is the number of individuals in each category. a) Individuals (men plus women) with lower aromatase expression had increased probability of survival compared to those with higher aromatase expression (P=0.0080). b) Women with lower aromatase expression have increased probability of survival compared to those with higher aromatase expression (P=0.0092). c) Aromatase expression provided no predictive power for survival for men with NSCLC (P=0.182). d) Women who were age 65 years and older and had lower aromatase expression were found to have an increased probability of survival compared to those with higher tumor aromatase expression (P=0.0061).

FIGS. 5a-d. Aromatase expression levels predict probability of survival in women with earlier (Stage I and II) NSCLC. All panels show Kaplan-Meier survival plots for individuals with NSCLC. Solid lines show lower aromatase expression (<1.5 mean integrated intensity) and dashed lines show higher aromatase expression (>1.5 mean integrated intensity). N is the number of individuals in each category. a) Individuals (men plus women) with Stage I/II NSCLC and lower aromatase expression have increased probability of survival compared to those with higher tumor aromatase expression (P=0.0048). b) Women with Stage I/II NSCLC and lower aromatase expression have increased probability of survival as compared to those with higher tumor aromatase expression (P=0.0102). c) Aromatase expression provided no predictive power for survival for men with Stage I/II NSCLC (P=0.152). d) Women with Stage I/II NSCLC who were age 65 years and older and who have lower aromatase expression have an increased probability of survival compared to those with higher tumor aromatase expression (P=0.0080).

FIGS. 6a-b. Kaplan-Meier survival plots for women with and without a history of smoking. Both panels show Kaplan-Meier survival plots for women with NSCLC. Solid lines show lower aromatase expression (<1.5 mean integrated intensity) and dashed lines show higher aromatase expression (>1.5 mean integrated intensity). N is the number of women in each category. a) Women (all ages and stages) with NSCLC who had a smoking history and have lower aromatase expression have a slight, but not statistically significant, increased probability of survival compared to those with higher aromatase expression (P=0.063). b) Women (all ages and stages) with NSCLC who had no history of ever smoking and have lower aromatase expression have an increased probability of survival compared to those with higher tumor aromatase expression (P=0.022). Note that although the population size was relatively small, 92% of the female non-smokers with lower tumor aromatase survived beyond 5-years.

FIG. 7. Aromatase expression levels predict probability of survival in women 65 years of age and older with Stage I and II NSCLC. A Kaplan-Meier survival plot for individuals with NSCLC is displayed. Solid lines show lower aromatase expression (≦1.35 mean integrated intensity) and dashed lines show higher aromatase expression (>1.35 mean integrated intensity). N is the number of individuals in each category. Women with Stage I/II NSCLC who were age 65 years and older and who have lower aromatase expression have increased probability of survival compared to those with higher tumor aromatase expression (P=0.0002). In this sub-population, at 5 years, 86% of women with lower aromatase survived while only 53% of women with higher tumor aromatase survived.

FIG. 8. Aromatase expression in a patient cohort from MD Anderson Center predict probability of survival in women 65 years of age and older with Stage I and II NSCLC. A lung cancer TMA was obtained from MD Anderson Cancer Center and used for validation of the results obtained on the TMA constructed at UCLA. The patient population of the MD Anderson TMA is shown in Table 4. A Kaplan-Meier survival plot for individuals with NSCLC is displayed. Solid lines show lower aromatase expression (<0.45 mean integrated intensity) and dashed lines show higher aromatase expression (>0.45 mean integrated intensity). N is the number of individuals in each category. Women with Stage I/II NSCLC who were age 65 years and older and who have lower aromatase expression have increased probability of survival compared to those with higher tumor aromatase expression (P=0.005).

### DETAILED DESCRIPTION OF THE INVENTION

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

Abbreviations: NSCLC, non-small cell lung cancer; ER, estrogen receptor; IHC, immunohistochemistry; TMA, tissue microarray; DAB, diaminobenzidine.

Estrogen signaling is critical in the progression of tumors that bear estrogen receptors. In most patients with breast cancer, inhibitors that block interactions of estrogen with its receptors or suppress the production of endogenous estrogens, are important interventions in the clinic. Recent evidence now suggests that estrogen also contributes to the pathogenesis of non-small cell lung cancer.

As discussed in detail below, we used a human lung cancer xenograph model system, to analyze the effect of aromatase or estradiol on tumor growth. We further examined the level of protein expression of aromatase in 422 non-small cell lung cancer (NSCLC) patients using a high-density tissue microarray. Results were confirmed and validated on an independent patient cohort (n=337). Lower levels of aromatase predicted a greater chance of survival in women 65 years and older. Within this population, the prognostic value of aromatase was greatest in earlier (stage I/II) lung cancer. In addition, for women with no history of smoking, lower aromatase levels were a strong predictor of survival. These findings identify aromatase as an early stage predictor of survival in some women with NSCLC. These studies also provide evidence that that targeted treatment of women whose lungs cancers have higher levels of aromatase, will be good candidates for treatment with an aromatase inhibitors.

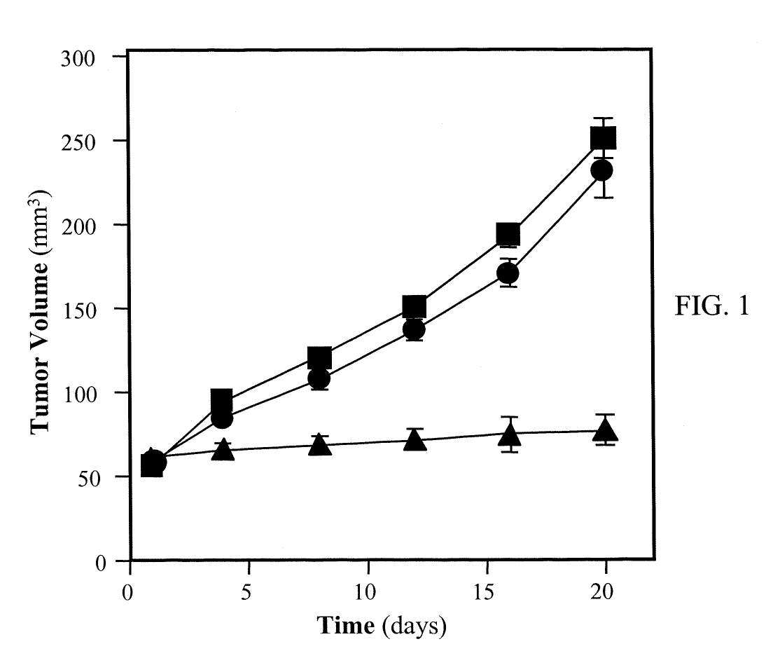

Previous studies suggested that increased local estrogen levels and/or enhanced stimulation of the ER signaling pathway may play a role in lung cancer progression (see, e.g. Stabile et al., Cancer Res 2002; 62:2141-50; Stabile et al., Cancer Res 2005; 65:1459-70; Pietras et al., Steroids 2005; 70:372-81, Weinberg et al., Cancer Res 2005; 65:11287-91). In this study we examined aromatase, a key enzyme in the estrogen synthesis pathway. We hypothesized that expression of aromatase in lung cancer cells could result in a focal increase in estrogen levels and serve to promote tumor development and/or progression. Recently, we have demonstrated that aromatase is expressed and is active in human lung cells (see, e.g. Weinberg et al., Cancer Res 2005; 65:11287-91) and that an aromatase inhibitor reduces cell growth in vitro and lung tumor xenograph growth in nude mice (see, e.g. Weinberg et al., Cancer Res 2005; 65:11287-91). To further assess estrogen signaling in NSCLC in vivo, we implanted A549 lung tumor cells which express endogenous aromatase in ovariectomized female athymic mice. As shown in FIG. 1, mice treated with androstenedione, a substrate of aromatase, showed significantly enhanced tumor growth compared to untreated control animals (P<0.001). Moreover, treatment with estradiol, the ligand for estrogen receptor, showed a similar effect on enhancing lung tumor growth (FIG. 1).

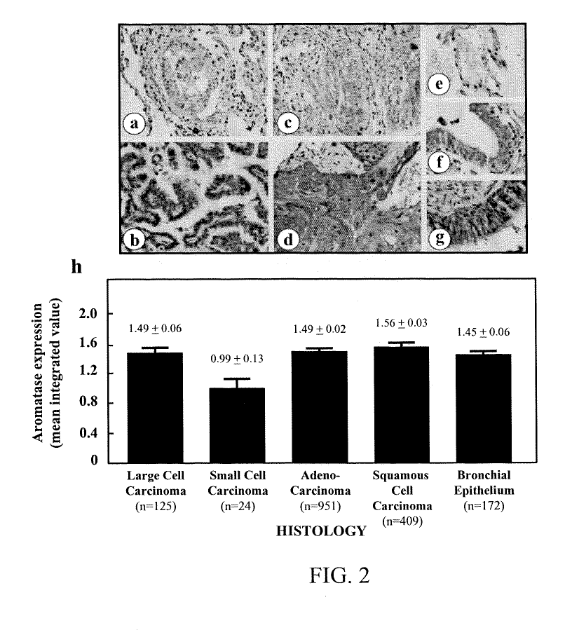

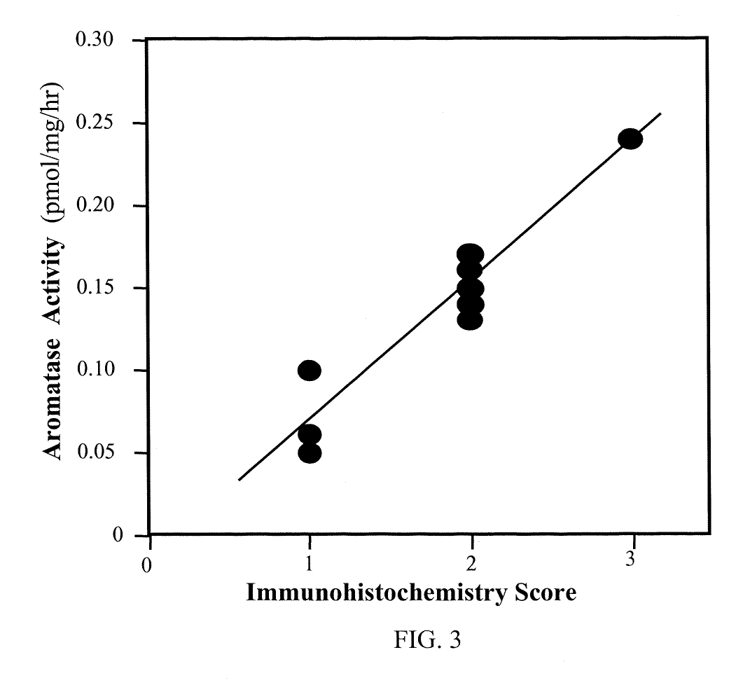

Based on the results above as well as earlier studies showing enhanced levels of aromatase expression and activity in NSCLC cell lines and human tumor samples (see, e.g. Weinberg et al., Cancer Res 2005; 65:11287-91), we initiated a study to examine in situ expression of aromatase on a population basis using a high-density lung tissue microarray (TMA) consisting of malignant and benign clinical samples (the clinical and pathologic patient categories of the TMA are shown in Table 2). Protein expression levels were determined by immunohistochemical staining. In this study, 1,681 TMA spots were stained representing 422 surgical cases. Aromatase displayed a cytoplasmic localization with patterns ranging from below the level of detection (score of 0.0) to abundant (score of 3.0) (FIG. 2a-g). Moreover, aromatase staining intensity as determined by immunohistochemistry, correlated well with aromatase enzyme activity as assessed by a radioassay (FIG. 3). Aromatase staining was observed in all tumor subtypes as well as non-neoplastic bronchial/bronchiolar epithelium. On a per-spot basis, mean integrated staining intensities (a measure of intensity and cellular frequency; see e.g. the Examples) were similar among adenocarcinomas, squamous cell carcinomas, large cell carcinomas and non-neoplastic bronchial epithelium (FIG. 2h).

Specific discoveries relating to the correlation between the level of aromatase expression in a specific NSCLC and, for example, the prognosis for that malignancy are discussed in the sections below.

### Aromatase Expression Predicts Disease Outcome in Women

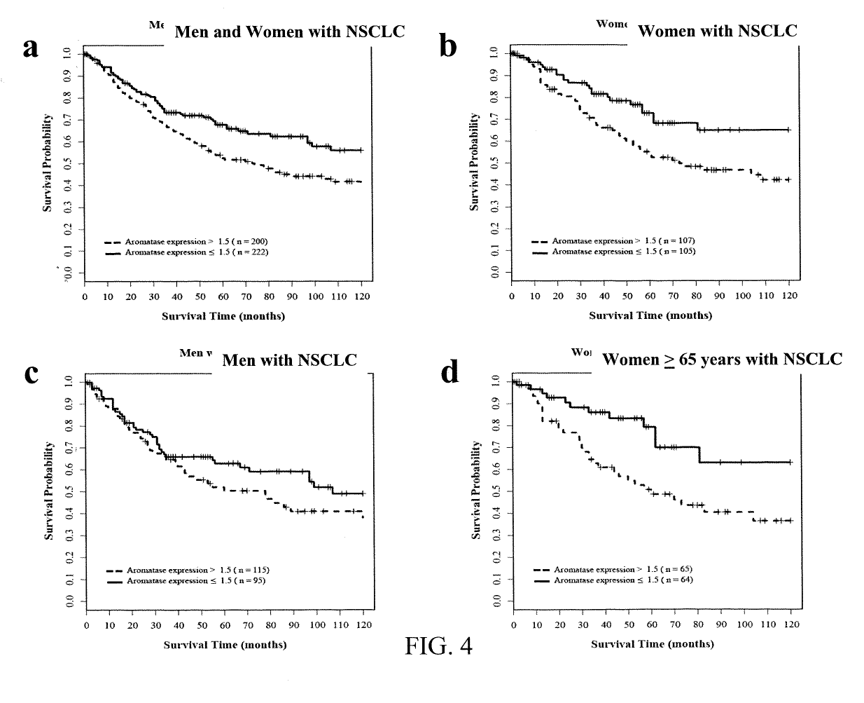

Despite the lack of significant differences of aromatase staining on a per-spot basis, there was patient-to-patient diversity in the staining pattern. Therefore, we examined whether aromatase expression levels offered any predictive value for patient survival compared to death due to disease. We first examined aromatase expression as a continuous variable. Using the univariate Cox proportional hazards model, aromatase expression approached significance as a predictor of survival (P=0.056, hazards ratio 1.28, 95% confidence level 0.994-1.65). To consider whether aromatase expression was predictive for a subset of the population, we next studied a dichotomized population. We initially chose the midpoint integrated intensity (1.5 on a scale of 0.0-3.0) to define “higher” and “lower” expression of aromatase (see Example section below). Coincidentally, 1.5 was also the median value for aromatase integrated expression in the patient population. As shown in FIG. 4a, there was a slight survival advantage for individuals with lower levels of aromatase expression compared to individuals with higher levels (P=0.008). Significantly, upon dividing the population by gender, the predictive value of aromatase expression was observed for women (FIG. 4b; P=0.009) but not for men (FIG. 4c; P=0.182). This observation did not appear to be due to differences in the cellular expression or activity of aromatase in the lungs in women compared to men (see, e.g. Weinberg et al., Cancer Res 2005; 65:11287-91 and Table 3).

We further stratified the female population according to age (e.g., age cut offs of 50, 55, 60, 65, and 70 years) and examined the predictive capability of aromatase expression in each age group. Aromatase levels were not predictive of disease outcome in women younger than 65 years of age with lung cancer. However, as shown in FIG. 4d, women 65 years and older had a striking survival advantage if they expressed lower levels of aromatase (P=0.006), with 79% of these women surviving 5 years post surgery compared to only 49% survival in the population of women with higher aromatase.

### Aromatase Expression is an Early-Stage Predictive Tumor Marker

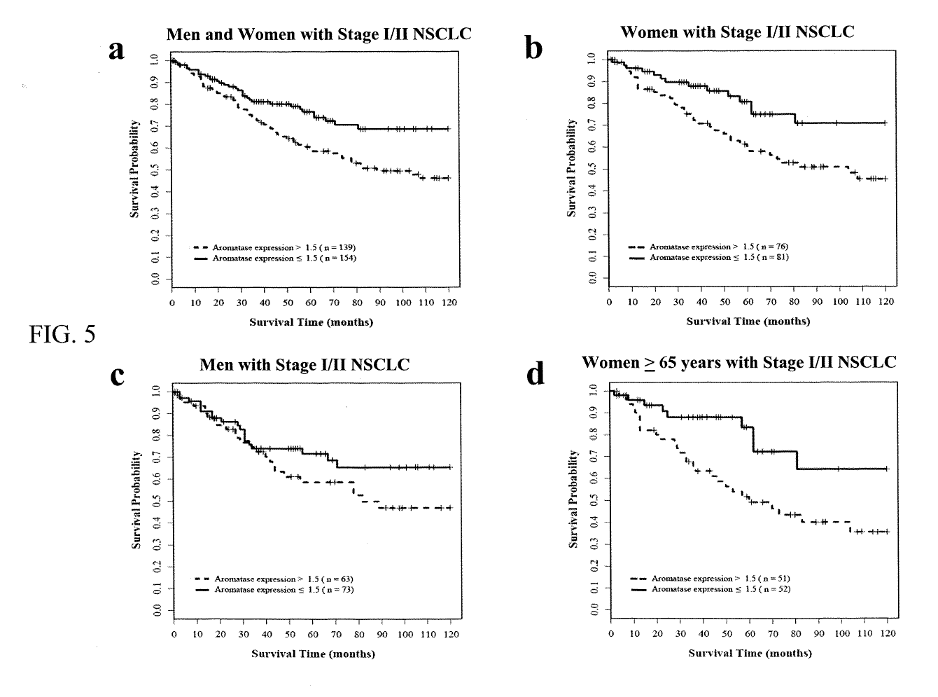

We next subdivided the population based on disease stage. For individuals with stage III or IV NSCLC, aromatase expression levels did not display significant association with disease outcome. In contrast, for patients of all gender with earlier stage lung cancer (Stage I and II), lower levels of aromatase predicted greater survival (FIG. 5a; P=0.005). Furthermore, this predictive value was gender-specific with low aromatase predicting greater survival probability in women with Stage I/II (FIG. 5b; P=0.010) but not men (FIG. 5c; P=0.152). Of the 157 cases of women who were afflicted with Stage I and II NSCLC, the 5-year survival among the population with lower aromatase (n=81) was 81%, while the survival of women with higher aromatase expression (n=76) was 60% (FIG. 5b). Again, the predictive value of aromatase expression was far more pronounced in women 65 years and older (FIG. 5d; P=0.008) than in younger women. Internal validation of survival predictions, performed as described using 400 bootstrapped samples (see, e.g. Jemal et al., CA Cancer J Clin 2006; 56:106-30; and Kim et al., J Urol 2005; 173:1496-501), guaranteed that data was not overfit.

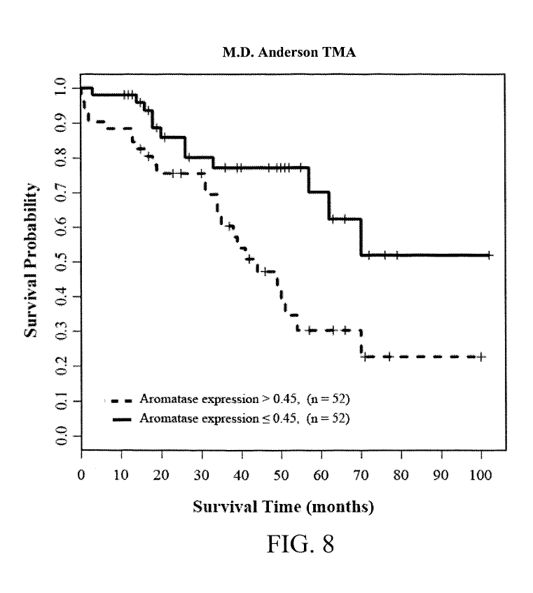

To further validate the findings, an additional independent set of NSCLC patients was examined. We examined aromatase expression using a TMA constructed from patients at the University of Texas MD Anderson Cancer Center (Table 4). This set consisted of 337 patients with a breakdown of 183 women (104 age 65 or over, 79 under age 65) and 154 men. The median staining intensity was used to divide the patient groups as described above. Significantly, an identical pattern was identified for the predictive power of aromatase levels and survival in women >65 years old with early stage NSCLC completely consistent with results from the UCLA patient cohort (P=0.005; FIG. 8).

Aromatase Expression and NSCLC in Women with No Smoking History

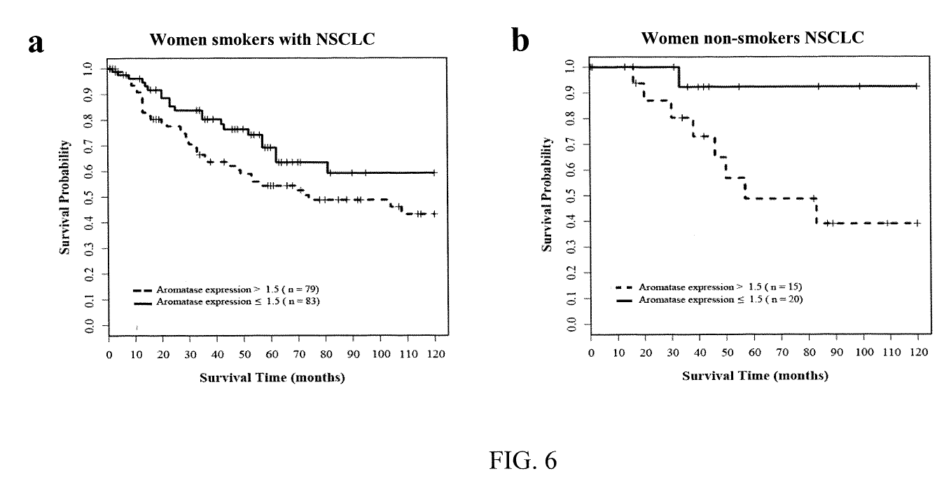

The population of women with NSCLC was further analyzed in order to identify any associations based on smoking history. Women were dichotomized into those who had smoked >100 cigarettes at any point in their lifetime (smokers), and those who had never smoked (non-smokers) (see Methods). Within the population of women (all ages) who had smoked during their lifetime, there was a slightly reduced—albeit not statistically significant—predicted survival advantage with lower levels of aromatase (FIG. 6a; P=0.063). Although in this particular patient cohort the subset of women who had no smoking history yet developed NSCLC was relatively small, there was still a highly significant predictive value for survival with lower tumor aromatase levels (FIG. 6b; P=0.022) with a 5 year survival percentage of 92% (n=20) compared to a 49% survival percentage for women with higher tumor aromatase (n=15).

Finally, we addressed whether aromatase expression had predictive information beyond other known NSCLC prognostic indicators such as tumor stage, tumor grade, and patient age. In multivariate Cox proportional hazards models, aromatase as either a continuous (P=0.0015, Table 1) or dichotomized (P=0.0042) variable was a significant independent predictor of survival for women of age 65 and older.

As noted above, aromatase expression levels are an early-stage predictive biomarker of lung cancer survival. Specifically, lower relative levels of aromatase predict a higher probability of survival in women with NSCLC who are 65 years of age and older. The predictive power of aromatase expression was particularly informative at earlier stages of lung cancer (stage I and II). In contrast, women with higher aromatase expression, have a worse prognosis.

For malignancies such as breast cancer, stimulation of the ER pathway contributes to the pathogenesis of this disease (see, e.g. Yager et al., N Engl J Med 2006; 354:270-82). The pathogenic effect of estrogen is thought to be a by-product of estrogen metabolism and/or estrogen-induced gene transcription promoting proliferation and inhibition of apoptosis (see, e.g. Yager et al., N Engl J Med 2006; 354:270-82). It is now appreciated that the normal lung is also responsive to estrogen for regulating differentiation and development (see, e.g. Stabile et al., Cancer Res 2002; 62:2141-50). We and others have hypothesized that increased local estrogen levels and/or enhanced stimulation of the ER signaling pathway also plays a role in lung cancer progression (see, e.g. Stabile et al., Cancer Res 2002; 62:2141-50; Stabile et al., Cancer Res 2005; 65:1459-70; Pietras et al., Steroids 2005; 70:372-81, Weinberg et al., Cancer Res 2005; 65:11287-91). As part of this concept, we predict that lung cancer cells may adopt one or more strategies to maintain or enhance the ER signaling pathway, including the local production of estrogens via aromatase-catalyzed biosynthesis.

It is intriguing that aromatase expression levels are most predictive in a later age in women. That this is purely due to diminishing levels of circulating estrogen in postmenopausal women is unlikely since the median age for menopause is approximately 51.4 years. However, circulating levels of androgens (e.g., androstenedione and testosterone), the substrate for aromatase, remain relatively constant in women throughout menopausal changes, and gradually decrease after age 65 (see, e.g. Adly et al., International journal of cancer 2006; 119:2402-7; Cappola et al., The Journal of clinical endocrinology and metabolism 2007; 92:509-16; Davison et al., The Journal of clinical endocrinology and metabolism 2005; 90: 3847-53; Burger et al., The Journal of clinical endocrinology and metabolism 2000; 85:2832-8; Djahanbakhch et al., The Journal of pathology 2007; 211:219-31). Therefore, we predict that in such situations where systemic levels of estrogen are relatively decreased due to declined estrogen produced by the ovaries (see, e.g. Folkerd et al., J Steroid Biochem Mol Biol 2006; 102:250-5) and androgen levels are decreasing, tumor cells and/or neighboring host cells may compensate by increasing endogenous production of estrogen through aromatase. Growth factor receptor signaling pathways may also contribute to increased expression and activity of aromatase, thereby further stimulating estrogen biosynthesis in NSCLC cells (see, e.g. Subbaramaiah et al., Cancer Res 2006; 66:5504-11). In contrast to these results, we observed no predictive value for aromatase expression levels in men of any age. Of note, estrogen levels in men stay relatively constant with age (see, e.g. Vermeulen et al., Aging Male 2002; 5:98-102). Thus, it is possible that in men with lung cancer, an alternate strategy is employed to enhance ER pathway signaling

An interesting yet surprising finding was the association between aromatase levels and survival differences in women with NSCLC and no smoking history. In this subpopulation, over 90% of women who had lower aromatase expression, survived beyond 5 years. It is intriguing to consider that in addition to allowing a predictive stratification of NSCLC patients, these results may also suggest novel therapeutic approaches to treat NSCLC using currently available aromatase inhibitors (e.g. Arimidex®, Aromasin®, and Femara®). Aromatase inhibitors are showing promising results in malignancies such as breast cancer (see, e.g. Smith et al., N Engl J Med 2003; 48:2431-42; Coombes et al., N Engl J Med 2004; 350:1081-92; Gould et al., Curr Opin Obstet Gynecol 2006; 18:41-6). Of note, a recent trial was conducted where postmenopausal women with primary breast cancer either remained on tamoxifen or were switched to a third generation aromatase inhibitor, exemestane (see, e.g. Coombes et al., N Engl J Med 2004; 350:1081-92). Significantly, there was a reduction in the subsequent development of primary lung cancer in the cohort on exemestane compared to those women maintained on tamoxifen (see, e.g. Coombes et al., N Engl J Med 2004; 350:1081-92). Thus, a compelling prediction of the current study is that aromatase inhibitors may be a beneficial addition to the armamentarium of compounds currently being used to treat and possibly prevent lung cancer.

In the studies disclosed herein, we hypothesized that lung cancer progression was hormonally influenced with a key player in growth promotion being estrogen. The data from studies that is presented herein is consistent with this mechanism. These studies include a population-based examination of aromatase levels in human non-small cell lung cancers (NSCLC) using Tissue Microarray technology combined with sophisticated bioinformatics analytical approaches. Findings from this study (as confirmed on two independent patient cohorts) include for example the observations that: (1) low aromatase expression levels are a strong predictor of survival (versus death) in women with NSCLC (and potentially in other variants of lung cancer); (2) the predictive power of the aromatase expression profile is strongest in earlier (stage I/II) lung cancer; and (3) low aromatase expression levels are also a predictor of survival in women with NSCLC who have no smoking history. The data presented herein further provides evidence that the use of aromatase inhibitors might be affective in women with NSCLC in a manner akin to the ways in which these inhibitors function in other cancers (various aromatase inhibitors are currently FDA-approved and in use to treat diseases such as breast cancer).

### Illustrative Embodiments of the Invention and their Uses

Currently, there are no effective biomarkers (protein, DNA, mRNA, or carbohydrate) which can predict the course of disease and outcome for individuals with Non Small Cell Lung Cancer (NSCLC). Over 160,000 individuals die from lung cancer annually; NSCLC is the most common type of lung cancer. Moreover, other than surgery, there are no effective treatment for NSCLC.

In this context, embodiments of the invention solve this problem by identifying aromatase as an early biomarker detector/predictor of survival in a major subset of individuals with NSCLC. As disclosed herein, the term “aromatase”, is used according to its art accepted meaning and refers to, for example, the gene whose nucleic acid sequence is shown in SEQ ID NO. 1 and whose amino acid sequence is shown in SEQ ID NO. 2. Embodiments of the invention examine the expression of the aromatase gene in non-small cell lung cancers. As is known in the art, non-small cell lung cancer is the most common kind of lung cancer, with non-small cell lung cancers referring to a group of lung cancers named for the kinds of cells found in the cancer and how the cells look under a microscope. The three main types of non-small cell lung cancer are squamous cell carcinoma, large cell carcinoma, and adenocarcinoma. The cells in these sub-types differ in size, shape, and chemical make-up. Squamous cell carcinoma: About 25% to 30% of all lung cancers are this kind. They are linked to smoking and tend to be found in the middle of the lungs, near a bronchus. Adenocarcinoma: This type accounts for about 40% of lung cancers. It is usually found in the outer part of the lung. Large-cell (undifferentiated) carcinoma: About 10% to 15% of lung cancers are this type. It can start in any part of the lung. It tends to grow and spread quickly, which makes it harder to treat.

Embodiments of this invention can be used to provide predictive information about the survival of women with NSCLC who have no or little smoking history. Moreover, embodiments of this invention can be used to direct a rational therapeutic intervention strategy for women with NSCLC through, for example, the use of FDA-approved aromatase inhibitors. Conceivably, other agents which inhibit the biosynthesis, degradation, binding and/or action of estrogen in the lung can similarly be used. Patient benefits from embodiments of this invention include, but are not limited to: predicting the disease course of lung cancer and predicting the probability of long-term versus short-term survival time. Based on such survival predictions, embodiments of the this invention can then assist physicians in determining the aggressiveness of subsequent therapy and/or surgical intervention. For example, embodiments of the invention are useful for predicting the responsiveness of patients with lung cancer to aromatase inhibitors or other agents which inhibit the biosynthesis, degradation, binding and/or action of estrogen in the lung.

Embodiments of the this invention could readily be translated into a variety of clinically appropriate tests. For example, expression levels of aromatase (or other genes/proteins related to estrogen binding or signaling) could be determined by methods which include, but are not limited to 1) immuno-staining of tissue; 2) Western blot analysis; 3) Northern blot analysis; 4) PCR; 5) ELISA of cellular extracts. In addition, it is possible that aromatase is released into the blood and that levels of this protein could be measured by protocols similar to or identical to ELISA and/or enzyme activity could be measured. Typical embodiments of the invention can be broken down into steps which typically include methodological elements such as identifying a woman suspected of lung cancer who is 65 years or older; performing a biopsy or surgical resection of tissue; formalin-fixing, paraffin embedded tissue and prepare tissue sections on slide (standard histological process) or Freeze tissue sample in OCT and prepare a frozen section of tissue (standard histological process). One can then for example, stain tissue with Goat-anti-Human aromatase antibody C-16 (Santa Cruz Biotechnology) using standard immunohistochemistry techniques.

There are a number of different embodiments of the invention that are based on the observation that the level of aromatase polypeptide expressed by a NSCLC cell can be correlated with factors such as the probability of the patient surviving for some period of time, for example at least 1, 2, 3, 4 or 5 years from the time of the examination. In this context, a typical embodiment of the invention is a method of examining a human non-small cell lung carcinoma obtained from a patient comprising the steps of observing the level of aromatase (SEQ ID NO: 2) polypeptide expressed in the non-small cell lung carcinoma, wherein the patient is female and is at least 65 years old. In certain embodiments of the invention, a low level of aromatase expression identifies the patient as having a greater probability of surviving for at least 1, 2, 3, 4 or 5 years from the time of the examination than does a patient identified as having a high level of aromatase expression.

Certain embodiments of the invention further comprise the step of using the observed level of aromatase (SEQ ID NO: 2) polypeptide expressed in the non-small cell lung carcinoma to determine a prognosis for the patient. In typical embodiments of the invention, the prognosis comprises an estimate of the probability of the patient surviving for at least 6 months, 1, 2, 3, 4, 5, 6, 7, 8, 9 or 10 years from the time of the examination. Other embodiments of the invention comprise the step of using the observed level of aromatase (SEQ ID NO: 2) polypeptide expressed by the non-small cell lung carcinoma to evaluate a course of therapy for the patient (e.g. whether to operate or not, the optimal therapeutic agent or agents etc.). In certain embodiments of the invention, a high level of aromatase expression identifies the patient as a candidate for treatment with an aromatase inhibitor. Optionally in such embodiments, a course of therapy for a patient exhibiting such an aromatase profile includes administering one or more aromatase inhibitors to the patient.

A number of other factors can be considered in the diagnostic and prognostic methods of the invention. For example, in addition to factors such as age and gender, environmental factors such as a person's behavior can be considered. In one such embodiment of the invention, a methodological step includes determining if the patient has a lifetime history of smoking more than 100 cigarettes. In addition, in certain embodiments of the invention, the methods which examine aromatase expression are combined with other methods commonly used in cancer diagnoses and prognosis. For example in one such embodiment of the invention, a tissue sample (e.g. from a biopsy) is examined to determine the stage or grade of cancer cells in the same (if any). In typical embodiments of the invention, one examines a sample having or suspected of containing non-small cell lung carcinoma cells to observe the presence (or absence) of a stage I, stage II, stage III or stage IV non-small cell lung carcinoma.

Certain embodiments of the invention involve the determination of the stage of the lung cancer. Lung cancer staging is an assessment of the degree of spread of the cancer from its original source. It is an important factor affecting the prognosis and potential treatment of lung cancer. Non-small cell lung carcinoma is staged from IA (“one A”, best prognosis) to IV (“four”, worst prognosis). Small cell lung carcinoma is classified as limited stage if it is confined to one half of the chest and within the scope of a single radiotherapy field. Otherwise it is extensive stage. Such stages are described in detail in a variety of publications, for example: Mountain, C F; Libshitz H I, Hermes K E (2003) “A Handbook for Staging, Imaging, and Lymph Node Classification” Charles P Young Ed., the contents of which are incorporated by reference.

A number of methods for examining aromatase expression can be used in embodiments of the invention. In some embodiments of the invention, the level of aromatase polypeptides expressed by a non-small cell lung carcinoma are observed using an assay of aromatase polypeptide enzymatic activity, for example by using the radiolabeled substrate, [1β-3H]androst-4-ene-3,17-dione (Perkin-Elmer, Boston, Mass.) as taught in the art (see, e.g. Weinberg et al., Cancer Res 2005; 65:11287-91). Alternatively, a level of aromatase polypeptide expressed by the non-small cell lung carcinoma is observed using an antibody that binds aromatase polypeptide. In one such embodiment of the invention, the antibody is used in an immunohistochemical assay of cytoplasmic aromatase staining intensity; the assay comprises a numerical scale of staining intensity values including a mean or median staining intensity value; a staining intensity value above the mean or median staining intensity value characterizes the cell as having a high level of aromatase expression; and a staining intensity value below the mean or median staining intensity value characterizes the cell as having a low level of aromatase expression. In a specific illustrative embodiment of this, the antibody is used in an immunohistochemical assay of cytoplasmic aromatase staining intensity using a value scale of 0-3; a staining intensity value of 0 corresponds to an amount of aromatase staining that is below the level of detection; a staining intensity value of 1 corresponds to a weak amount of aromatase staining; a staining intensity value of 2 corresponds to a moderate amount of aromatase staining; and a staining intensity value of 3 corresponds to a strong amount of aromatase staining; a staining intensity value above 1.5 characterizes the cell as having a high level of aromatase expression; and a staining intensity value below 1.5 characterizes the cell as having a low level of aromatase expression. Aromatase antibody staining intensity can be qualified and/or quantified via a number of methods known in the art. For example, in some embodiments of the invention, a numerical scale of aromatase antibody staining intensity values (e.g. a median or mean intensity value) is derived from a plurality of stained cells (e.g. noncancerous and cancerous cell) having a plurality of levels of aromatase expression. In certain embodiments of the invention, the level of aromatase polypeptide expressed by the non-small cell lung carcinoma is observed using a high-density tissue microarray. Optionally in the methods of the invention, the staining intensity value is quantified using image analysis software.

As noted above, in some embodiments of the invention, aromatase polypeptide is measured directly, for example by using an enzymatic assay or an antibody that binds aromatase. In other embodiments of the invention, the level of aromatase polypeptide expressed in the non-small cell lung carcinoma is observed indirectly by using a polymerase chain reaction to observe the level of aromatase mRNA in the cell that encode the aromatase polypeptide; and correlating the level of aromatase mRNA observed in the cell with the level of aromatase polypeptide expressed in the cell. In particular, because it is known in the art that levels of aromatase enzymatic activity correlate with levels of aromatase protein and/or levels of aromatase mRNA (see, e.g. Chen et al., Endocrine—Related Cancer (1999) 6: 149-156; and Weinberg et al., Cancer Res. 2005; 65: (24) 11287-11291, the contents of which are incorporated herein by reference), certain embodiments of the invention examine aromatase polynucleotides as an indirect measurement of aromatase polypeptide expression and activity.

In one such embodiment of the invention, the level of aromatase polypeptide expressed in the non-small cell lung carcinoma is observed by: observing the level of aromatase mRNA in the cell that encode the aromatase polypeptide; and correlating the level of aromatase mRNA observed in the cell with the level of aromatase polypeptide expressed in the cell. Optionally in such methods, the aromatase mRNA is observed in the cell using a polynucleotide primer or probe that hybridizes to the aromatase mRNA. Such methods can be combined with further methodological steps, for example, the step of determining if a cell in the tissue sample is a stage I/II non-small cell lung carcinoma. Alternatively such steps are used to obtain information useful for determining a prognosis or therapy for a patient of a specific gender and/or age category and/or specific behavioral history, by for example, determining if the patient has a lifetime history of smoking more than 100 cigarettes; or determining the age of the patient or determining the gender of the patient.

Another embodiment of the invention is a method of obtaining information useful for determining a prognosis or therapy for a female patient having or suspected of having a human non-small cell lung carcinoma, the method comprising observing a level of aromatase (SEQ ID NO: 2) polypeptide expressed in a tissue sample obtained from the patient that includes or is suspected of including the human non-small cell lung carcinoma cells; and then correlating the level of aromatase polypeptide observed in the cells with a prognosis or therapy for non-small cell lung carcinoma that is associated with the observed level of aromatase (SEQ ID NO: 2) polypeptide expressed by the cells. In certain embodiments of the invention, the level of aromatase polypeptide expressed in the tissue sample is determined using an immunohistochemical staining technique; and the method further comprises determining the percentage of cells in the tissue sample that are stained in the immunohistochemical staining technique. Optionally in such methods, the level of aromatase polypeptide expressed in the tissue sample is determined using an immunohistochemical staining technique; the method is preformed on a plurality of patients; and the plurality of patients are stratified into those having a staining intensity in the immunohistochemical staining technique that is either above or below a mean or median staining intensity value on a numerical scale of staining intensity values derived from a plurality of stained cells having a plurality of levels of aromatase expression. Optionally in these methods, the stratification is used to predict the probability of the patient surviving for at least 1, 2, 3, 4 or 5 years from the time of the examination; and/or identify patients likely to respond to a therapeutic regimen comprising a aromatase inhibitor.

One specific embodiment of the invention comprises examining aromatase expression in lung cancer tissue cells using immunohistochemistry or immunofluorescence. A typical first step is to identify a woman suspected of lung cancer. A typical next step comprises performing a biopsy or surgical resection of tumor. A typical next step is to formalin-fix, paraffin embed tissue and prepare tissue sections on slide (standard histological process) or Freeze tissue sample in OCT and prepare a frozen section of tissue (standard histological process). In a typical next step, incubated tissue sections are incubated in antigen retrieval buffer at 95° C. for 30 min for antigen retrieval (see, e.g. the Examples below). In a typical next step endogenous peroxidases are quenched using hydrogen peroxidase, and nonspecific binding was blocked for 5 min. After rinsing, slides are incubated with goat anti-human aromatase antibody at a 1:100 dilution for 1 hr at room temperature (one vendor who offers this antibody is Santa Cruz Biotechnology). Antibody binding can be detected using the Dako Envision System with diaminobenzidine or a similar system. Alternatively, fluorescent-tagged antibodies can be used. Typically in such methods, the sections are counterstained. Negative controls are typically performed on sections using the same protocol as above except with non-immune goat IgG replacing a primary antibody. Typically in such methods, tissues with known levels of aromatase expression are stained simultaneously and serve as standards. In some embodiments, a “standard” to which an unknown sample is compared to examine if aromatase levels are high or low is derived from established tissue or cells lines which express high or low levels of aromatase. In certain embodiments, the percentage of cancer cells stained plus the intensity of staining of each cancer cell is then quantified for example using a scale of 0-3 by either a pathologist or by image analysis hardware and software. As a final step, patient staining intensity data can then be stratified into those above or below the mean staining of the standard controls. In addition to aromatase, one can measure the expression levels of other proteins involved in estrogen synthesis, metabolism, binding, and/or signal transduction. Aromatase levels in men does not appear to predict survival from NSCLC. Potentially, the expression levels and/or activity of other proteins involved in estrogen synthesis, metabolism, binding, and/or signal transduction may be of prognostic/diagnostic value in men and/or aromatase expression may be predictive in variants of lung cancer other than NSCLC.

As used herein, the terms “hybridize”, “hybridizing”, “hybridizes” and the like, used in the context of polynucleotides, are meant to refer to conventional hybridization conditions. “Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, are exemplified by: (1) hybridization in 50% formamide, 2×SSC, 0.1% SDS, 10 mg/ml salmon sperm DNA, and 10% dextran sulfate, at 42° C. for 16 hours followed by a washing in 2×SSC, 0.1% SDS at 25° C. for 10 min (three times), and washed in the same solution at 65° C. for 5 min (twice) and are generally identified by, but not limited to, those that: (2) employ conditions of low ionic strength and high temperature for washing, for example 0.015 M sodium chloride/0.0015 M sodium citrate/0.1% sodium dodecyl sulfate at 50° C.; (3) employ during hybridization a denaturing agent, such as formamide, for example, about 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (4) employ 50% formamide, about 2-5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium. citrate) and 50% formamide at 55° C., followed by a high-stringency wash consisting of 0.1×SSC containing EDTA at 55° C.

### Illustrative Methods for Observing Aromatase Expression

Oncogenesis is known to be a multistep process where cellular growth becomes progressively dysregulated and cells progress from a normal physiological state to precancerous and then cancerous states (see, e.g., Alers et al., Lab Invest. 77(5): 437-438 (1997) and Isaacs et al., Cancer Surv. 23: 19-32 (1995)). In this context, examining a biological sample for evidence altered gene expression (such as aberrant aromatase expression in lung cancers) can allow artisans to obtain useful information (e.g. diagnostic and prognostic information). In such examinations, the status of aromatase polypeptide in a biological sample of interest can be compared to a standard or control, for example, or to the status of aromatase in a corresponding normal sample (e.g. a sample from that individual or alternatively another individual that is not affected by a pathology) and/or a population of NSLC cells obtained from various patients. An alteration in the status of aromatase expression in the biological sample (as compared to a control or standardized sample and/or value) provides evidence of dysregulated cellular growth.

In addition to using normal cells as a comparative sample or aromatase expression, one can alternatively use a predetermined normative value such as a predetermined normal level of aromatase mRNA or polypeptide expression (see, e.g., Greyer et al., J. Comp. Neurol. 1996 Dec. 9; 376(2):306-14 and U.S. Pat. No. 5,837,501) to evaluate levels of aromatase expression in a given sample. The term “status” in this context is used according to its art accepted meaning and refers to the condition or state of a gene and its products. Typically, skilled artisans use a number of parameters to evaluate the condition or state of a gene and its products. These include, but are not limited to the location of expressed gene products (including the location of aromatase expressing cells) as well as the level, and biological activity of expressed gene products (such as aromatase mRNA, polynucleotides and polypeptides).

A wide variety of methods known in the art can be used to examine the expression of aromatase polypeptides and polynucleotides in cells such as non-small cell lung carcinomas. For example, certain embodiments of methods which examine aromatase polynucleotides and polypeptides in such cells are analogous to those methods from well-established diagnostic assays known in the art such as those that observe the activity of molecules such as PSA polynucleotides and polypeptides. For example, just as PSA polynucleotides are used as probes (for example in Northern analysis, see, e.g., Sharief et al., Biochem. Mol. Biol. Int. 33(3):567-74 (1994)) and primers (for example in PCR analysis, see, e.g., Okegawa et al., J. Urol. 163(4): 1189-1190 (2000)) to observe the presence and/or the level of PSA mRNAs in methods of monitoring PSA expression or the metastasis of prostate cancers, the aromatase polynucleotides described herein can be utilized in the same way to detect aromatase underexpression or the metastasis of lung and other cancers having an alteration in the expression of this gene. Similarly, just as PSA polypeptides are used to generate antibodies specific for PSA which can then be used to observe the presence and/or the level of PSA proteins in methods to monitor PSA protein expression (see, e.g., Stephan et al., Urology 55(4):560-3 (2000)) in prostate cells (see, e.g., Alanen et al., Pathol. Res. Pract. 192(3):233-7 (1996)), the aromatase polypeptides described herein can be utilized to generate antibodies for use in detecting aromatase expression in lung cells (as well as cells from other cancer lineages observed to have an alteration in the expression of this gene).

Another aspect of the present invention relates to methods for detecting aromatase polynucleotides and aromatase-related proteins, as well as methods for identifying a cell that expresses aromatase. The expression profile of aromatase can make it a diagnostic marker for aberrant cell growth. Accordingly, the status of aromatase gene products provides information useful for predicting a variety of factors including susceptibility to advanced stage disease, rate of progression, and/or tumor aggressiveness. As discussed in detail herein, the status of aromatase gene products in patient samples can be analyzed by a variety protocols that are well known in the art including immunohistochemical analysis, the variety of Northern blotting techniques including in situ hybridization, RT-PCR analysis (e.g. quantitative RT-PCR), Western blot analysis, tissue array analysis and the like.

The invention provides assays for the evaluation of aromatase polynucleotides in a biological sample, such lung, and other tissues, cell preparations, and the like. Aromatase polynucleotides which can be evaluated include, for example, a aromatase gene or fragment thereof, aromatase mRNA, alternative splice variant aromatase mRNAs, and recombinant DNA or RNA molecules that contain a aromatase polynucleotide. A number of methods for amplifying and/or detecting the presence of aromatase polynucleotides are well known in the art and can be employed in the practice of this aspect of the invention. In one embodiment, a method for detecting an aromatase mRNA in a biological sample comprises producing cDNA from the sample by reverse transcription using at least one primer; amplifying the cDNA so produced using an aromatase polynucleotides as sense and antisense primers to amplify aromatase cDNAs therein; and detecting the presence of the amplified aromatase cDNA. Optionally, the sequence of the amplified aromatase cDNA can be determined.

In another embodiment, a method of detecting a aromatase gene in a biological sample comprises first isolating genomic DNA from the sample; amplifying the isolated genomic DNA using aromatase polynucleotides as sense and antisense primers; and detecting the presence of the amplified aromatase gene. Any number of appropriate sense and antisense probe combinations can be designed from the nucleotide sequence provided for the aromatase and used for this purpose.

The invention also provides assays for detecting the presence of an aromatase protein in a tissue or other biological sample such as lung and other tissues, and the like. Methods for detecting a aromatase-related protein are also well known and include, for example, immunoprecipitation, immunohistochemical analysis, Western blot analysis, molecular binding assays, ELISA, ELIFA and the like. For example, a method of detecting the presence of a aromatase-related protein in a biological sample comprises first contacting the sample with a aromatase antibody, a aromatase-reactive fragment thereof, or a recombinant protein containing an antigen binding region of a aromatase antibody; and then detecting the binding of aromatase-related protein in the sample. Optionally, aromatase polypeptides are measured high-denisty tissue microarray.

Methods for identifying a cell that overexpresses or under expresses aromatase polypeptides are exemplary embodiments of the invention. In one embodiment, an assay for identifying a cell that quantifies the expression of the aromatase gene comprises detecting the presence of aromatase mRNA concentrations in the cell. Methods for the evaluation of particular mRNAs in cells are well known and include, for example, hybridization assays using complementary DNA probes (such as in situ hybridization using labeled aromatase riboprobes, Northern blot and related techniques) and various nucleic acid amplification assays (such as RT-PCR using complementary primers specific for aromatase, and other amplification type detection methods, such as, for example, branched DNA, SISBA, TMA and the like). Alternatively, an assay for identifying a cell that expresses a aromatase gene comprises evaluating the presence of aromatase-related protein in the cell. Various methods for the detection of proteins are well known in the art and are employed for the detection of aromatase-related proteins and cells that express aromatase-related proteins.

As noted above, the status of aromatase expression in a tissue sample (e.g. one harboring or suspected of harboring NSLC cells) can be analyzed by a number of means well known in the art, including without limitation, immunohistochemical analysis, in situ hybridization, RT-PCR analysis (e.g. on laser capture micro-dissected samples), Western blot analysis, and tissue array analysis. Typical protocols for evaluating the status of the aromatase gene and gene products are found, for example in Ausubel et al. eds., 1995, Current Protocols In Molecular Biology, Units 2 (Northern Blotting), 4 (Southern Blotting), 15 (Immunoblotting) and 18 (PCR Analysis). Thus, the status of aromatase in a biological sample is evaluated by various methods utilized by skilled artisans including, but not limited to genomic Southern analysis (to examine, for example perturbations in the aromatase gene), Northern analysis and/or PCR analysis of aromatase mRNA (to examine, for example alterations in the polynucleotide sequences or expression levels of aromatase mRNAs), and, Western and/or immunohistochemical analysis (to examine, for example alterations in polypeptide sequences, alterations in polypeptide localization within a sample, alterations in expression levels of aromatase proteins and/or associations of aromatase proteins with polypeptide binding partners). Detectable aromatase polynucleotides include, for example, a aromatase gene or fragment thereof, aromatase mRNA, alternative splice variants, aromatase mRNAs, and recombinant DNA or RNA molecules containing a aromatase polynucleotide.

In another embodiment of the invention, one can evaluate the status aromatase nucleotide and amino acid sequences in a biological sample in order to identify perturbations in the structure of these molecules. These perturbations can include insertions, deletions, substitutions and the like in the coding and regulatory regions of the aromatase gene. Such evaluations are useful because perturbations in the nucleotide and amino acid sequences are observed in a large number of proteins associated with a growth dysregulated phenotype (see, e.g., Marrogi et al., 1999, J. Cutan. Pathol. 26(8):369-378). For example, a mutation in the sequence of an aromatase 5′ regulatory enhancer and/or promoter sequence may provide evidence of a tumor. Such assays therefore have diagnostic and predictive value where a mutation in aromatase indicates a potential loss of function or increase in tumor growth.

A wide variety of assays for observing perturbations in nucleotide and amino acid sequences are well known in the art. For example, the size and structure of nucleic acid or amino acid sequences of aromatase gene products are observed by the Northern, Southern, Western, PCR and DNA sequencing protocols discussed herein. In addition, other methods for observing perturbations in nucleotide and amino acid sequences such as single strand conformation polymorphism analysis are well known in the art (see, e.g., U.S. Pat. Nos. 5,382,510 issued 7 Sep. 1999, and 5,952,170 issued 17 Jan. 1995).

Additionally, one can examine the methylation status of the aromatase gene in a biological sample. Aberrant demethylation and/or hypermethylation of CpG islands in gene 5′ regulatory regions frequently occurs in immortalized and transformed cells, and can result in altered expression of various genes. For example, promoter hypermethylation of the pi-class glutathione S-transferase (a protein expressed in normal prostate but not expressed in >90% of prostate carcinomas) appears to permanently silence transcription of this gene and is the most frequently detected genomic alteration in prostate carcinomas (De Marzo et al., Am. J. Pathol. 155(6): 1985-1992 (1999)). In addition, this alteration is present in at least 70% of cases of high-grade prostatic intraepithelial neoplasia (PIN) (Brooks et al, Cancer Epidemiol. Biomarkers Prev., 1998, 7:531-536). In another example, expression of the LAGE-I tumor specific gene (which is not expressed in normal prostate but is expressed in 25-50% of prostate cancers) is induced by deoxy-azacytidine in lymphoblastoid cells, suggesting that tumoral expression is due to demethylation (Lethe et al., Int. J. Cancer 76(6): 903-908 (1998)). A variety of assays for examining methylation status of a gene are well known in the art. For example, one can utilize, in Southern hybridization approaches, methylation-sensitive restriction enzymes which cannot cleave sequences that contain methylated CpG sites to assess the methylation status of CpG islands. In addition, MSP (methylation specific PCR) can rapidly profile the methylation status of all the CpG sites present in a CpG island of a given gene. This procedure involves initial modification of DNA by sodium bisulfite (which will convert all unmethylated cytosines to uracil) followed by amplification using primers specific for methylated versus unmethylated DNA. Protocols involving methylation interference can also be found for example in Current Protocols In Molecular Biology, Unit 12, Frederick M. Ausubel et al. eds., 1995.

The invention also comprises methods for gauging tumor aggressiveness. In one embodiment, a method for gauging aggressiveness of a tumor comprises determining the level of aromatase mRNA or aromatase protein expressed by tumor cells, comparing the level so determined to the level of aromatase mRNA or aromatase protein expressed in a corresponding normal tissue taken from the same individual or a normal tissue reference sample, wherein the degree of aromatase mRNA or aromatase protein expression in the tumor sample relative to the normal sample indicates the degree of aggressiveness. In a specific embodiment, aggressiveness of a tumor is evaluated by determining the extent to which aromatase is expressed in the tumor cells, with higher expression levels indicating more aggressive tumors. Another embodiment is the evaluation of the integrity of aromatase nucleotide and amino acid sequences in a biological sample, in order to identify perturbations in the structure of these molecules such as insertions, deletions, substitutions and the like. The presence of one or more perturbations indicates more aggressive tumors.

Another embodiment of the invention is directed to methods for observing the progression of a malignancy in an individual over time. In one embodiment, methods for observing the progression of a malignancy in an individual over time comprise determining the level of aromatase mRNA or aromatase protein expressed by cells in a sample of the tumor, comparing the level so determined to the level of aromatase mRNA or aromatase protein expressed in an equivalent tissue sample taken from the same individual at a different time, wherein the degree of aromatase mRNA or aromatase protein expression in the tumor sample over time provides information on the progression of the cancer. In a specific embodiment, the progression of a cancer is evaluated by determining aromatase expression in the tumor cells over time, where an increased expression over time indicates a progression of the cancer. Also, one can evaluate the integrity aromatase nucleotide and amino acid sequences in a biological sample in order to identify perturbations in the structure of these molecules such as insertions, deletions, substitutions and the like, where the presence of one or more perturbations indicates a progression of the cancer.

The above diagnostic approaches can be combined with any one of a wide variety of prognostic and diagnostic protocols known in the art. For example, another embodiment of the invention is directed to methods for observing a coincidence between the underexpression of aromatase gene and aromatase gene products (or perturbations in aromatase gene and aromatase gene products) and a factor that is associated with malignancy, as a means for diagnosing and prognosticating the status of a tissue sample. A wide variety of factors associated with malignancy can be utilized, such as the expression of genes associated with malignancy (e.g. RB, p53, p16 for lung cancer etc.) as well as gross cytological observations (see, e.g., Bocking et al., 1984, Anal. Quant. Cytol. 6(2):74-88; Epstein, 1995, Hum. Pathol. 26(2):223-9; Thorson et al., 1998, Mod. Pathol. 11 (6):543-51; Baisden et al., 1999, Am. J. Surg. Pathol. 23(8):918-24). Methods for observing a coincidence between the expression of aromatase gene and aromatase gene products (or perturbations in aromatase gene and aromatase gene products) and another factor that is associated with malignancy are useful, for example, because the presence of a set of specific factors that coincide with disease provides information crucial for diagnosing and prognosticating the status of a tissue sample.

Methods for detecting and quantifying the expression of aromatase mRNA or protein are described herein, and standard nucleic acid and protein detection and quantification technologies are well known in the art. Standard methods for the detection and quantification of aromatase mRNA include in situ hybridization using labeled aromatase riboprobes, Northern blot and related techniques using aromatase polynucleotide probes, RT-PCR analysis using primers specific for aromatase, and other amplification type detection methods, such as, for example, branched DNA, SISBA, TMA and the like. In a specific embodiment, semi-quantitative RT-PCR is used to detect and quantify aromatase mRNA expression. Any number of primers capable of amplifying aromatase can be used for this purpose, including but not limited to the various primer sets specifically described herein. In a specific embodiment, polyclonal or monoclonal antibodies specifically reactive with the wild-type aromatase protein can be used in an immunohistochemical assay of biopsied tissue.

As illustrated above, the status of aromatase gene may be evaluated by a variety of methods well known in the art. The evaluation of the status of the aromatase gene provides information useful in diagnostic and prognostic protocols which assess cells which may have disregulated growth. In typical embodiments, the invention consists of methods of detecting evidence of disregulated growth in a cell such as a cell suspected of being cancerous, by examining the status of the aromatase gene by any of a number of art accepted protocols such as a genomic Southerns to evaluate gross perturbations of genomic DNA, Northern and PCR analysis to evaluate the levels aromatase mRNAs or immunological methods to examine aromatase proteins. Such protocols are used to examine the level of aromatase expression as well as the presence or absence of mutations within the aromatase mRNA or proteins. In this context, these methods are used to compare the status of aromatase in the test cell to the status of aromatase gene in a corresponding normal cell or to a specific known standard, where an alteration in the status of aromatase gene in the test cell relative to the normal cell provides evidence of disregulated growth within the test cell.

Yet another embodiment of the invention is a method of examining a test biological sample comprising a human lung cell for evidence of altered cell growth, the method comprising evaluating the levels of aromatase polynucleotides in the biological sample, wherein an increase in the levels of aromatase polynucleotides in the test sample relative to a normal tissue sample provide evidence of altered cell growth; and wherein the levels of aromatase polynucleotides in the cell are evaluated by contacting the sample with a polynucleotide probe (including primers utilized in PCR analyses) that specifically hybridizes to a aromatase nucleotide sequence shown in SEQ ID NO: 1 and evaluating the presence of a hybridization complex formed by the hybridization of the polynucleotide probe with aromatase polynucleotides in the sample. In some embodiments of the invention the presence of a hybridization complex is evaluated by Northern analysis and/or polymerase chain reaction. In another aspect of this embodiment, these methods further include evaluating the test biological sample for the presence of an additional factor that is associated with altered cell growth (e.g. a decrease in the expression of a tumor suppressor gene known in the art such as Rb and/or an increase in the expression of an oncogene known in the art such as c-ras). In a specific embodiment of the invention, a decrease in the levels of aromatase polynucleotides in the test sample relative to a normal tissue sample provide evidence of lung cancer. In another specific embodiments of the invention, the aromatase polynucleotides in the test sample are mRNA.

Yet another embodiment of the invention is a method of examining a test biological sample comprising, for example, a human lung cell for evidence of altered cell growth, the method comprising evaluating the levels of aromatase polypeptides in the biological sample, wherein a increase in the levels of aromatase polypeptides in the test sample relative to a normal tissue sample provide evidence of altered cell growth; and wherein the levels of aromatase polypeptides in the cell are evaluated by contacting the sample with an antibody that immunospecifically binds to a aromatase polypeptide sequence shown in SEQ ID NO: 2 and evaluating the presence of a complex formed by the binding of the antibody with aromatase polypeptides in the sample. Yet another embodiment of the invention is a method of examining a test biological sample comprising, for example, a human lung cell for evidence of altered cell growth, the method comprising evaluating the levels of aromatase polypeptides in the biological sample, wherein a decrease in the levels of aromatase polypeptides in the test sample relative to a normal tissue sample provide evidence of altered cell growth; and wherein the levels of aromatase polypeptides in the cell are evaluated by contacting the sample with an antibody that immunospecifically binds to a aromatase polypeptide sequence shown in SEQ ID NO: 2 and evaluating the presence of a complex formed by the binding of the antibody with aromatase polypeptides in the sample. In these embodiments, the presence of a complex is typically evaluated by a method selected from the group consisting of ELISA analysis, Western analysis and immunohistochemistry.

Compositions useful in the methods disclosed herein typically include one or more antibodies that bind aromatase and which can be used as a probe to monitor aromatase polypeptide expression in a cell. Other compositions useful in the methods disclosed herein can include one or more aromatase nucleic acid molecules designed for use as a probe such as a PCR primer in a method used to monitor aromatase in a cell. Specifically contemplated nucleic acid related embodiments of the invention disclosed herein are genomic DNA, cDNAs, ribozymes, and antisense molecules, as well as nucleic acid molecules based on an alternative backbone, or including alternative bases, whether derived from natural sources or synthesized, and include molecules capable of inhibiting the RNA or protein expression of aromatase. For example, antisense molecules can be RNAs or other molecules, including peptide nucleic acids (PNAs) or non-nucleic acid molecules such as phosphorothioate derivatives, that specifically bind DNA or RNA in a base pair-dependent manner. A skilled artisan can readily obtain these classes of nucleic acid molecules using the aromatase polynucleotides and polynucleotide sequences disclosed herein.

For use in the methods described above, kits are also provided by the invention. Such kits may comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be an antibody or polynucleotide specific for aromatase protein or aromatase gene or message, respectively. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kits of the invention have a number of embodiments. A typical embodiment is a kit comprising a container, a label on said container, and a composition contained within said container; wherein the composition includes: (1) a polynucleotide that hybridizes to a complement of the aromatase polynucleotide shown in SEQ ID NO: 1; and/or (2) an antibody that binds the aromatase polypeptide shown in SEQ ID NO: 2, the label on said container indicates that the composition can be used to evaluate the presence of aromatase in at least one type of mammalian cell, and instructions for using the aromatase polynucleotide or antibody for evaluating the presence of aromatase RNA, DNA or protein in at least one type of mammalian cell.

### EXAMPLES

Various aspects of the invention are further described and illustrated by way of the several examples that follow, none of which are intended to limit the scope of the invention. Certain disclosure in the examples below can be found in Mah et al., Cancer Res. 2007 Nov. 1; 67(21):10484-90, the contents of which are incorporated by reference.

### Example

### Typical Materials and Methods Useful For Practicing Embodiments of the Invention

### Tumor Xenograph Studies

The protocol used for implantation of human lung tumor xenografts in nude mice was described previously (see, e.g. Weinberg et al., Cancer Res 2005; 65:11287-91; Pietras et al., Oncogene 1995; 10:2435-46). Fifteen 6-week old ovariectomized athymic female mice were injected subcutaneously with A549 cells (2×10⁸ cells/mouse) and subsequently treated with daily subcutaneous injections with androstenedione, estradiol, or buffer control (5 mice per group) as previously described (see, e.g. Weinberg et al., Cancer Res 2005; 65:11287-91; Pietras et al., Oncogene 1995; 10:2435-46).

### Aromatase Activity

NSCLC specimens were procured under an IRB-approved protocol from 7 males and 5 females. Half of each specimen was snap frozen at −70° C. and half was formalin-fixed, processed, and embedded in paraffin. Aromatase activity was assessed on the frozen aliquots by radioassay and IHC was performed on formalin-fixed, paraffin-embedded sections (see below). Activity of the enzyme was measured using the radiolabeled substrate, [1β-3H]androst-4-ene-3,17-dione (Perkin-Elmer, Boston, Mass.) as previously reported (see, e.g. Weinberg et al., Cancer Res 2005; 65:11287-91). Aromatase activity was then correlated with protein expression by IHC using linear regression analysis (see, e.g. Weinberg et al., Cancer Res 2005; 65:11287-91).

### Lung Tissue Microarray

A lung tissue microarray (TMA) was constructed under appropriate IRB and HIPAA regulations, using archival formalin-fixed, paraffin-embedded lung tissue samples from the Department of Pathology and Laboratory Medicine, at the UCLA Medical Center as previously described (see, e.g. Kononen et al., Nat Med 1998; 4:844-7). The TMA contains tissue from 696 surgical specimens from 671 patients. Of these samples, 422 were marker-informative cases linked to outcome information (survival versus death due to disease) for individuals with NSCLC. Sampled tissues included primary lung tumor, matched non-neoplastic lung parenchyma, and metastatic lung carcinoma to lymph nodes and distant sites. Sections of all blocks that were used were reviewed by a board-certified pathologist to confirm the diagnosis. At least three core tissue biopsies, each 0.6 mm in diameter, were taken from select, morphologically representative regions of each paraffin embedded lung tumor and precisely arrayed using a custom-built instrument, as previously described (see, e.g. Kononen et al., Nat Med 1998; 4:844-7; Seligson et al., Int J Oncol 2005; 27:131-41; Seligson et al., Nature 2005; 435:1262-6; Shi et al., Mod Pathol 2005; 18:547-57). Table 2 summarizes the patient cohort used in this study. The demographics, histopathological distribution, grade, stage, clinical parameters, smoking history, and outcome (survival, and death due to disease) of the population studied here were similar to those reported in the United States (see, e.g. Jemal et al., CA Cancer J Clin 2006; 56:106-30) (American Cancer Society. Cancer Facts & Figures 2006; American Lung Association, Trends in Lung Cancer Morbidity and Mortality, May 2005; National Cancer Institute, SEER Cancer Statistics Review, 1975-2003). Individuals who were classified as “nonsmokers” had no history of ever smoking cigarettes. Individuals classified as “smokers” had all smoked >100 cigarettes at one point in their life.

### Immunohistochemistry

Lung TMA blocks were sectioned immediately prior to being stained. The protocol for slide preparation and section staining has previously been described (see, e.g. Weinberg et al., Cancer Res 2005; 65:11287-91; Seligson et al., Nature 2005; 435:1262-6) Goat anti-human aromatase (CYP19A) antibody C-16 (# SC-14245) was purchased from Santa Cruz Biotechnology (Santa Cruz, Calif.). The entire lung TMA was stained using a standard two-step indirect immunohistochemistry protocol (see, e.g. Weinberg et al., Cancer Res 2005; 65:11287-91). Briefly, TMA sections were incubated in 0.01M sodium citrate buffer (pH 6.0) at 95° C. for 30 min for antigen retrieval. Endogenous peroxidases were quenched using 3% hydrogen peroxidase, and nonspecific binding was blocked with Background Sniper (Biocare Medical, Concord, Calif.) for 5 min. After rinsing, slides were incubated with anti-aromatase antibody (1:100 dilution) for 1 hr at room temperature. Antibody binding was detected using the Dako Envision System with diaminobenzidine (DAB). The sections were counterstained with Harris's haematoxylin. Negative controls were performed on identical TMA sections using the same protocol as above except with non-immune goat IgG replacing the primary antibody. A semi-quantitative analysis of the aromatase-stained lung TMA was performed on relevant malignant cells or normal lung epithelium by a board-certified pathologist (VM) blinded to clinical information, and spot-checked by a second pathologist (DBS). Cytoplasmic aromatase staining per array spot was determined based on staining intensity (0=below the level of detection, 1, weak; 2, moderate; and 3, strong) and the percentage of cells staining at each intensity level (0-100%) (see. e.g. Seligson et al., Int J Oncol 2005; 27:131-41). A final integrated value of intensity and frequency was derived using the following formula: [(3x)+(2y)+(1z)]/100 where x, y, and z are staining at intensity 3, 2, and 1, respectively. This value was then used to compare tissue staining.

### Statistical Analysis

Analyses were performed using freely available R software (http://www.R-project.org) including survival and rpart packages. Pooling criteria are discussed in the Supplemental material section. Aromatase expression differences among various subgroups was determined using the Wilcoxon signed rank test or Kruskal-Wallis rank sum test. For dichotomized (high versus low) aromatase expression, the Fisher exact test or Pearson chi square test was used for analysis with categorical variables such as stage, grade and smoking history. Survival curves were calculated using the Kaplan-Meier method and comparisons were done using the log-rank test. The Cox proportional hazards model (univariate and multivariate) was used to determine the significance of various factors related to survival. The proportional hazards assumption was verified using Schoenfeld, martingale, and dfbeta residuals. LogRank and Fisher exact P-values were two-sided and a P<0.05 was considered significant.

The midpoint and median intensity of 1.5 was used to define low and high aromatase expression. This represents a conservative, non-biased decision which was used in order to prevent any artificial over-fitting of the data. However, in addition to this, using recursive partitioning, regression trees (rpart package), and plotting log-rank p-values versus hazard ratios, we independently determined the cut-point that gave optimal results for the relevant subpopulations discussed here. This value (1.3) is close to the midpoint value used here; relevant results and a discussion of this cut-point determination is presented in Supplemental material.

Internal validation to protect against overfitting of data was performed as described (see, e.g. Kim et al., J Urol 2005; 173:1496-501; Kim et al., Clin Cancer Res 2004; 10:5464-71).

### General Information on Lung TMA and Patient Samples

We constructed a high-density tissue microarray (TMA) from formalin-fixed, paraffin-embedded lung specimens of benign and tumor tissues from human lung obtained from surgical cases between 1985-2002 (Department of Pathology and Laboratory Medicine, UCLA Medical Center). In this study, 1,681 tissue spots were analyzed representing 422 informative cases. Of these cases, 212 were women, and 210 were men, with ages ranging from 26 to 88 years (mean age of 65 years for men and women). In this study we examined non-small cell lung cancers (NSCLC). The histo-pathological breakdown of the cases were as follows: 249 cases were adenocarcinoma (143 women, 106 men), 112 were squamous cell carcinoma (41 women, 71 men), 37 large cell undifferentiated carcinoma (17 women, 21 men). Using the AJCC staging criteria, 293 patients were Stage I/II (157 women, 136 men), 99 were Stage III/IV (41 women, 58 men). Only tissue from the initial primary surgical cases was evaluated.

Classification of lung carcinomas was based on The World Health Organization (WHO) histological classifications (Travis, W D et al. World Health Organization Classification of Tumours. Pathology and Genetics of Tumours of the Lung, Pleura, Thymus and Heart. IARC Press: Lyon 2004).

Tumor staging was according to the American Joint Committee on Cancer (AJCC) and the International Union Against Cancer (UICC) tumor-node-metastasis (TNM) classification system (Greene F L, Page D L, Fleming I D, et al, eds. AJCC Cancer Staging Manual. 6th ed. New York: Springer; 2002:223-240.; Sobin L H, Wittekind C, eds. UICC TNM Classification of Malignant Tumours. 6th ed. New York, N.Y.: Wiley-Liss; 2002:131-141). Surgical pathology was used for T stage determination. Clinical, radiographic and/or postoperative pathologic information was used to determine N and M stages.

Table 2 shows the distribution of patients with respect to different clinicopathologic variables when separated into high or low aromatase expression. Hormone replacement therapy status was not available for women in this study. The endpoint of the study was disease-specific death. The survival time is given in months and represents the period from disease diagnosis to death. Patients alive at last follow-up or those with deaths not due to disease were censored at last follow-up. Death of unknown cause were excluded.

### Pooling Criteria

In order to arrive at a single expression value across multiple informative tumor tissue spots in each case, the mean integrated value was calculated. NSCLC histologies included in the population survival analyses were adenocarcinoma, large cell carcinoma, squamous cell carcinoma, adenosquamous carcinoma, adenoid cystic carcinoma and mucoepidermoid carcinoma. Excluded in the analyses were tissue spots representing any other histology and/or distant metastases.

### Determination of Optimal Cut-Points

In the immunohistochemical staining studies disclosed herein, we identify a midpoint cut-point of 1.5 for the staining intensity value on a numerical scale of staining intensity. This value is also the median value for aromatase integrated expression for the population. This value was used to minimize potential artificial overfitting of the data. In addition, splitting the population at the midpoint of expression, could be more amenable to potential clinical applications. As disclosed herein, this cut-point yielded highly significant results.

In addition to utilizing the mid-point, we also determined the optimal cut-point for these data. Thus, the robustness of the aromatase expression cut-point was examined by testing cut-off values across the full spectrum of potential aromatase intensities using 0.01 intervals from 0.0-3.0 using protocols similar to those described (see, e.g. Camp et al., Clin Cancer Res 10, 7252-9 (2004); and Dolled-Filhart, M. et al. Cancer Res 66, 5487-94 (2006)). The optimal cut-point for dichotomized integrated intensity values was determined to be at 1.3; at this value, there was a maximum hazard ratio and a minimal P-value.

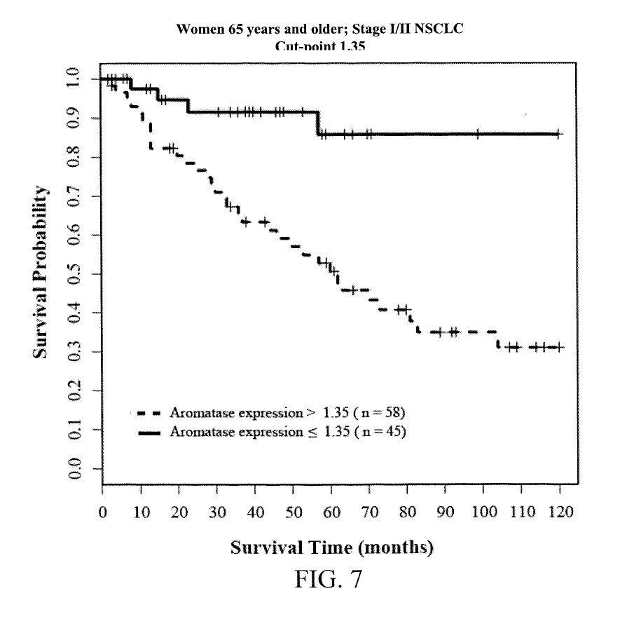

Notably, use of the optimal cut-point of 1.3 yielded a conclusion identical to that presented herein; the only difference was that lower P-values (i.e., higher significance) was obtained for all the survival analyses. A representative example is shown in FIG. 7. Nevertheless, despite the even greater significance using 1.3, we opted to present the more conservative data using a cut-off of 1.5 for the stated reasons above.

TABLE 1
Multivariate Cox proportional hazards analysis for women
65 years and older (n = 129)

| Variable | Hazard ratio | 95% CI | P |
|---|---|---|---|
| Tumor stage | 1.82 | 1.30-2.56 | 0.0005 |
| Tumor grade | 1.02 | 0.70-1.47 | 0.9300 |
| Age | 1.08 | 1.02-1.14 | 0.0054 |
| Aromatase | 2.37 | 1.39-4.04 | 0.0015 |
| mean intensity | | | |

TABLE 2
Clinico-pathologic characteristics and aromatase expression group membership in men and women with NSCLC.

| | Women ≧65 years | | | Women <65 years | | | Men | | |
|---|---|---|---|---|---|---|---|---|---|
| | Aroma- | Aroma- | p- | Aroma- | Aroma- | p- | Aroma- | Aroma- | p- |
| Characteristics | tase >1.5^{a} | tase ≦1.5^{b} | value | tase >1.5^{a} | tase ≦1.5^{b} | value | tase >1.5^{a} | tase ≦1.5^{b} | value |
| Total | 65 | 64 | | 40 | 43 | | 96 | 115 | |
| Age^{c} | 72.2 (65-88) | 72.3 (65-86) | 0.82^{e} | 54.0 (34-64) | 54.8 (39-64) | 0.78^{e} | 66.5 (26-87) | 63.9 (33-83) | 0.11^{e} |
| Stage | | | 0.30^{f} | | | 0.81^{f} | | | 0.43^{f} |
| Stage I & II^{d} | 51 (78%) | 52 (81%) | | 25 (63%) | 29 (67%) | | 63 (66%) | 73 (63%) | |
| Stage III & | 11 (17%) | 6 (9%) | | 12 (30%) | 12 (28%) | | 23 (24%) | 35 (30%) | |
| IV^{d} | | | | | | | | | |
| Grade | | | 0.36^{f} | | | 0.25^{f} | | | 1.0^{f} |
| Grade (1 & 2)^{d} | 29 (45%) | 34 (53%) | | 17 (43%) | 20 (47%) | | 40 (42%) | 51 (44%) | |
| Grade (3 & 4)^{d} | 31 (49%) | 25 (39%) | | 23 (58%) | 15 (35%) | | 49 (52%) | 60 (52%) | |
| Smoking | | | 0.48^{f} | | | 1.0^{f} | | | 0.75^{f} |
| Smoking | 48 (74%) | 52 (81%) | | 31 (78%) | 31 (72%) | | 77 (81%) | 100 (87%) | |
| history^{d} | | | | | | | | | |
| Non-smoking | 13 (20%) | 9 (14%) | | 7 (18%) | 6 (14%) | | 5 (5%) | 5 (4%) | |
| history^{d} | | | | | | | | | |
| NSCLC | | | 0.15^{g} | | | 0.78^{g} | | | 0.32^{g} |
| Histology | | | | | | | | | |
| Adenocarcinoma^{d} | 39 (60%) | 42 (66%) | | 28 (70%) | 34 (79%) | | 48 (51%) | 58 (50%) | |
| Squamous | 17 (26%) | 14 (22%) | | 6 (15%) | 4 (9%) | | 31 (33%) | 40 (35%) | |
| cell carcinoma^{d} | | | | | | | | | |
| Large cell | 8 (12%) | 3 (5%) | | 3 (8%) | 3 (7%) | | 8 (8%) | 13 (11%) | |
| carcinoma^{d} | | | | | | | | | |
| Other^{d,h} | 1 (2%) | 5 (8%) | | 3 (8%) | 2 (5%) | | 9 (9%) | 4 (3%) | |

^{a}Mean integrated intensity values of greater than 1.5 (out of a total of 3)
^{b}Mean integrated intensity values of less than or equal to 1.5 (out of a total of 3)
^{c}Mean (range) for total cases of NSCLC and all subsets.
^{d}Numbers in parentheses represent percentages for category totals.
^{e}P-value was determined by the Mann-Whitney test.
^{f}P-value was determined by the Fisher's Exact test.
^{g}P-value was determined by the Pearson's Chi Square test.
^{h}Tumors in this category are adenosquamous carcinoma, adenoid cystic carcinoma and mucoepidermoid carcinoma.

TABLE 3
Aromatase expression in clinico-pathologic subsets of NSCLC patients

| | Men | | Women ≧65 years | | Women <65 years | | |
|---|---|---|---|---|---|---|---|
| Group | Mean ± SEM^{a} | n | Mean ± SEM^{a} | n | Mean ± SEM^{a} | n | P^{b} |
| Total | 1.46 ± 0.04 | 210 | 1.50 ± 0.06 | 129 | 1.46 ± 0.06 | 83 | 0.71 |
| Stage | | | | | | | |
| Stage I/II | 1.50 ± 0.05 | 136 | 1.48 ± 0.07 | 103 | 1.44 ± 0.08 | 54 | 0.84 |
| Stage III/IV | 1.37 ± 0.09 | 58 | 1.71 ± 0.14 | 17 | 1.49 ± 0.13 | 24 | 0.16 |
| Grade | | | | | | | |
| Grade 1/2 | 1.47 ± 0.08 | 90 | 1.42 ± 0.08 | 63 | 1.37 ± 0.11 | 36 | 0.82 |
| Grade 3/4 | 1.47 ± 0.05 | 109 | 1.62 ± 0.09 | 56 | 1.61 ± 0.08 | 38 | 0.18 |
| Smoking | | | | | | | |
| Smokers | 1.46 ± 0.50 | 177 | 1.47 ± 0.06 | 100 | 1.49 ± 0.07 | 62 | 0.92 |
| Non-smokers | 1.36 ± 0.24 | 10 | 1.67 ± 0.16 | 22 | 1.53 ± 0.23 | 13 | 0.65 |
| Tumor Histology | | | | | | | |
| Adenocarcinoma | 1.43 ± 0.06 | 106 | 1.43 ± 0.70 | 81 | 1.41 ± 0.07 | 62 | 0.90 |
| Squamous cell | 1.52 ± 0.08 | 71 | 1.61 ± 0.12 | 31 | 1.54 ± 0.16 | 10 | 0.71 |
| carcinoma | | | | | | | |
| Large cell | 1.32 ± 0.08 | 21 | 1.85 ± 0.17 | 11 | 1.59 ± 0.24 | 6 | 0.03 |
| carcinoma | | | | | | | |

^{a}Mean integrated expression ± standard error of the mean
^{b}Kruskal-Wallis rank sum test (between gender and age subgroups)

TABLE 4
Clinico-pathologic characteristics of the patient cohort represented
in the validation TMA constructed at MD Anderson.

| | | Women | | |
|---|---|---|---|---|
| Group | Total | ≧65 years | <65 years | Men |
| Patient population | 337 | 104 | 79 | 154 |
| Stage | | | | |
| Stage I/II | 273 | 85 | 59 | 129 |
| Stage III/IV | 63 | 19 | 20 | 24 |
| Tumor Histology | | | | |
| Adenocarcinoma | 211 | 71 | 63 | 77 |
| Squamous cell carcinoma | 120 | 32 | 15 | 73 |
| Large cell carcinoma | 1 | 0 | 0 | 1 |

TABLE 5
Aromatase Sequences (see, e.g. GenBank
Accession No. NM_000103)

AROMATASE POLYNUCLEOTIDE
(SEQ ID NO: 1)
GGGAGTTTCTGGAGGGCTGAACACGTGGAGGCAAACAGGAAGGTGAAG
AAGAACTTATCCTATCAGGACGGAAGGTCCTGTGCTCGGGATCTTCCA
GACGTCGCGACTCTAAATTGCCCCCTCTGAGGTCAAGGAACACAAGAT
GGTTTTGGAAATGCTGAACCCGATACATTATAACATCACCAGCATCGT
GCCTGAAGCCATGCCTGCTGCCACCATGCCAGTCCTGCTCCTCACTGG
CCTTTTTCTCTTGGTGTGGAATTATGAGGGCACATCCTCAATACCAGG
TCCTGGCTACTGCATGGGAATTGGACCCCTCATCTCCCACGGCAGATT
CCTGTGGATGGGGATCGGCAGTGCCTGCAACTACTACAACCGGGTATA
TGGAGAATTCATGCGAGTCTGGATCTCTGGAGAGGAAACACTCATTAT
CAGCAAGTCCTCAAGTATGTTCCACATAATGAAGCACAATCATTACAG
CTCTCGATTCGGCAGCAAACTTGGGCTGCAGTGCATCGGTATGCATGA
GAAAGGCATCATATTTAACAACAATCCAGAGCTCTGGAAAACAACTCG
ACCCTTCTTTATGAAAGCTCTGTCAGGCCCCGGCCTTGTTCGTATGGT
CACAGTCTGTGCTGAATCCCTCAAAACACATCTGGACAGGTTGGAGGA
GGTGACCAATGAATCGGGCTATGTGGACGTGTTGACCCTTCTGCGTCG
TGTCATGCTGGACACCTCTAACACGCTCTTCTTGAGGATCCCTTTGGA
CGAAAGTGCTATCGTGGTTAAAATCCAAGGTTATTTTGATGCATGGCA
AGCTCTCCTCATCAAACCAGACATCTTCTTTAAGATTTCTTGGCTATA
CAAAAAGTATGAGAAGTCTGTCAAGGATTTGAAAGATGCCATAGAAGT
TCTGATAGCAGAAAAAAGACGCAGGATTTCCACAGAAGAGAAACTGGA
AGAATGTATGGACTTTGCCACTGAGTTGATTTTAGCAGAGAAACGTGG
TGACCTGACAAGAGAGAATGTGAACCAGTGCATATTGGAAATGCTGAT
CGCAGCTCCTGACACCATGTCTGTCTCTTTGTTCTTCATGCTATTTCT
CATTGCAAAGCACCCTAATGTTGAAGAGGCAATAATAAAGGAAATCCA
GACTGTTATTGGTGAGAGAGACATAAAGATTGATGATATACAAAAATT
AAAAGTGATGGAAAACTTCATTTATGAGAGCATGCGGTACCAGCCTGT
CGTGGACTTGGTCATGCGCAAAGCCTTAGAAGATGATGTAATCGATGG
CTACCCAGTGAAAAAGGGGACAAACATTATCCTGAATATTGGAAGGAT
GCACAGACTCGAGTTTTTCCCCAAACCCAATGAATTTACTCTTGAAAA
TTTTGCAAAGAATGTTCCTTATAGGTACTTTCAGCCATTTGGCTTTGG
GCCCCGTGGCTGTGCAGGAAAGTACATCGCCATGGTGATGATGAAAGC
CATCCTCGTTACACTTCTGAGACGATTCCACGTGAAGACATTGCAAGG
ACAGTGTGTTGAGAGCATACAGAAGATACACGACTTGTCCTTGCACCC
AGATGAGACTAAAAACATGCTGGAAATGATCTTTACCCCAAGAAACTC
AGACAGGTGTCTGGAACACTAGAGAAGGCTGGTCAGTACCCACTCTGG
AGCATTTCTCATCAGTAGTTCACATACAAATCATCCATCCTTGCCAAT
AGTGTCATCCTCACAGTGAACACTCAGTGGCCCATGGCATTTTATAGG
CATACCTCCTATGGGTTGTCACCAAGCTAGGTGCTATTTGTCATCTGC
TCCTGTTCACACCAGAGAACCAGGCTACAAGAGAAAAAGCAGAGGCCA
AGAGTTTGAGGGAGAAATAGTCGGTGAAGAAACCGTATCCATAAAGAC
CCGATTCCACCAAATGTGCTTTGAGAAGGATAGGCCTTCATTAACAAA
ATGTATGTCTGGTTCCCCAGTAGAGCTCTACTGCCTCAACCCAAGGGG
ATTTTTATGTCTGGGGCAGAAACACTCAAGTTGATTAGAAAGACCAGG
CCAATGTCAGGGTACCTGGGGCCAAACCCACCTGCTAGTGTGAATTAA
AGTACTTTAATTTTGTTTTCTGTGGAGGTGGAAAAGCAACATTCATAG
TCTTTGGAGAAATGCTTAGAAATTCAGCATTTGACCCTTGCTGTGAAT
TAAGCCCAATTAATTCCTGTTTGTCTACATATGATCTGTCTGTGGCAA
AAGTTTAATCAGAGGAAATTCTTTCCCAGTCTGTCGATTTATGCCTCA
GCCACTTGCCTGTGCTACAATTCATTGTGTTACCTGTAGATTCAGGTA
ATACAAACTATATATAATCATCAAGTAATACAAACTAATTTAGTAATA
GCCTGGGTTAAGTATTATTAGGGCCCTGTGTCTGCTGTAGAAAAAAAA
ATTCACATGATGCACTTCAAATTCAAATAAAAATCCTTTTGGCATGTT
CCCATTTTTGCTTAGCTCAATTAGTGTGGCTAACCAAGAGATAACTGT
AAATGTGACATTGATTTGCTCTTACTACAGCTTCAGTGATTGGGGGAG
GAAAAGTCCCAACCCAATGGGCTCAAACTTCTAAGGGGTACTCCTCTC
ATCCCCTTATCCTTCTCCCTCGACATTTTCTCCCTCTTTCTTCCCATG
ACCCCAAAGCCAAGGGCAACAGATCAGTAAAGAACGTGGTCAGAGTAG
AACCCCTGAAGTATTTTTTAATCCTACCTCAAAATTTAACAGTTACCT
GAGAGATTTAACATTATCTAGTTCATTGAATCATTGTATGTGGTCATG
GATAAATTGCACACCTTGGAATTCGCTTTCTAAAGGAAATCAAATGAA
TGGAGGAACTTTCCAAACACCACTTTACTTGTGTTATATAGCCAATAT
AACTATCTCTACTGAATGTCATTGAAAAACTAAAAAATTAAACTTATT
TACAAATAGGTAAATATTTGTCATTGAATCCATTGCCATCCCATTTGA
CTGTTCTTTTCATCCTACTGTCTAGTAATAAGCTGAGTATAAGATGAC
AGTGTAATCTCCCTGAAAGCAGGAGCTACTTTCTTTCTTTTGTAATCT
ATTTCCATCCCCATTTCCCTGTCCTGTCTCCCTGTATTCACTCCCAAG
CTCAGTTCTGAATAGACATTCCTGCTCAGAGATACTCCCAACTGATGC
AGAAACCAAATAAAGAGGTAGGTATTCCAAGAATTCAAGAATGGACAT
TAGTAAAGAATAAAACATTTATTTGAGCTTGGAATTATTTGGATCATC
TATATGGCCTAAAAATATATGGACTATGCCTGTGTACCTGAATACGTA
TGTAGTCAGGTCAAGACAATCATCCAAATAACTTAGACCCCTAAAAGC
AAGGCCAGGATTTGCAATTTAATGTGTCCCAATTAATTCACTTGAAAA
TTAGTAACACTCTGTTTACGTTGCCTCTGGCTGGAGCTGCATGGTGGA
AGAAGCCCAACTTTGGATCCATGTACTTCACCCATCCAATACTCTTGG
GACATTTATGTGTATTTTATCTGTATATATGAAGCCAATGTCTATGTC
TACACAGTCAAAGTGAAATGCATGTTTGATATAGCTGTACATAGATAT
CTATTTTGCAGGTACAAAAATATCCTGGGGGAAAACTGGGAGTGGAAG
GGTGGGGGGTGGGAGTGAGGGACATGGGGGAGGGACAGGAAGAGGAGA
AGTGTTGGTTTGAACGATCCAAGCAAACTCTCCCAGAATCAAATTACC
TGGGTAGTTGTTCAACTTTTCACTCTGCTTAGCCTGTATAGACAAACC
CCATATATTTGTAGAGGCTTGGCCTTGGAATTCTGGAATACCATTGGC
TTTTCAGTAGGCTGATGAACACATTTTGAAAATTCTATTATCTTCAGA
ATTTTGCCCCATTGTTAAGTGCTTAACCGTCACTCTTGAATGTGCAAT
GTGCTGTGGATTCCATTTTCATCAGTTCTGAAAGAACTGCAATGTGTA
AATTATCAGTGAAATGCATGCATATAAGGGCTCTATCATTATCAAATT
GTAAGGACAATTGTACCCTTCTATATCTTTGGGCATGCTAGACACCCC
CATGCCTTCATTGAGATCCCATTTTCCCCCTCTCAAGTGGAAAATAAT
CACATCCAGCAAGCTCTCTCATTATTGAGAAATACCATTTGGAAATTG
CCACTTTTTATTCCTAAGCAGCACCTTTCACTGTTCATGATGCTAATG
TTCCACAAAAGCATGTGCCATTGGCCCACTGAAGGATAGAGGGACCCT
TTTCAATCTATATCAGCTGGGCTCTGGGACTGAATCTCTCACCTATTC
TTGCAGAAAGACATACTAATTAAACCTTGTCAAAGTAAAAAA
AROMATASE POLYPEPTIDE
(SEQ ID NO: 2)
MVLEMLNPIHYNITSIVPEAMPAATMPVLLLTGLFLLVWNYEGTSSIP
GPGYCMGIGPLISHGRFLWMGIGSACNYYNRVYGEFMRVWISGEETLI
ISKSSSMFHIMKHNHYSSRFGSKLGLQCIGMHEKGIIFNNNPELWKTT
RPFFMKALSGPGLVRMVTVCAESLKTHLDRLEEVTNESGYVDVLTLLR
RVMLDTSNTLFLRIPLDESAIVVKIQGYFDAWQALLIKPDIFFKISWL
YKKYEKSVKDLKDAIEVLIAEKRRRISTEEKLEECMDFATELILAEKR
GDLTRENVNQCILEMLIAAPDTMSVSLFFMLFLIAKHPNVEEAIIKEI
QTVIGERDIKIDDIQKLKVMENFIYESMRYQPVVDLVMRKALEDDVID
GYPVKKGTNIILNIGRMHRLEFFPKPNEFTLENFAKNVPYRYFQPFGF
GPRGCAGKYIAMVMMKAILVTLLRRFHVKTLQGQCVESIQKIHDLSLH
PDETKNMLEMIFTPRNSDRCLEH

Throughout this application, various publications are referenced. The disclosures of these publications are hereby incorporated by reference herein in their entireties.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

What is claimed is:

1. A method of examining a human non-small cell lung carcinoma obtained from a patient comprising the steps of observing the level of aromatase (SEQ ID NO: 2) polypeptide expressed in the non-small cell lung carcinoma, wherein the patient is female and is at least 65 years old.

2. The method of claim 1, further comprising the step of using the observed level of aromatase (SEQ ID NO: 2) polypeptide expressed in the non-small cell lung carcinoma to determine a prognosis for the patient.

3. The method of claim 2, wherein the prognosis comprises an estimate of the probability of the patient surviving for at least 1, 2, 3, 4 or 5 years from the time of the examination.

4. The method of claim 1, further comprising the step of using the observed level of aromatase (SEQ ID NO: 2) polypeptide expressed by the non-small cell lung carcinoma to evaluate a course of therapy for the patient.

5. The method of claim 4, wherein a course of therapy for the patient includes administering one or more aromatase inhibitors to the patient.

6. The method of claim 1, further comprising the step of determining if the patient has a lifetime history of smoking more than 100 cigarettes.

7. The method of claim 1, further comprising determining if the non-small cell lung carcinoma is a stage I, stage II, stage III or stage IV non-small cell lung carcinoma.

8. The method of claim 1, wherein the level of aromatase polypeptide expressed by the non-small cell lung carcinoma is observed using an assay of aromatase polypeptide enzymatic activity.

9. The method of claim 1, wherein the level of aromatase polypeptide expressed by the non-small cell lung carcinoma is observed using an antibody that binds aromatase polypeptide.

10. The method of claim 9, wherein:
the antibody is used in an immunohistochemical assay of cytoplasmic aromatase staining intensity;
the assay comprises a numerical scale of staining intensity values including a mean or median staining intensity value;
a staining intensity value above the mean or median staining intensity value characterizes the cell as having a high level of aromatase expression; and
a staining intensity value below the mean or median staining intensity value characterizes the cell as having a low level of aromatase expression.

11. The method of claim 10, wherein:
the antibody is used in an immunohistochemical assay of cytoplasmic aromatase staining intensity using a value scale of 0-3;
a staining intensity value of 0 corresponds to an amount of aromatase staining that is below the level of detection;
a staining intensity value of 1 corresponds to a weak amount of aromatase staining;
a staining intensity value of 2 corresponds to a moderate amount of aromatase staining;
and a staining intensity value of 4 corresponds to a strong amount of aromatase staining;
a staining intensity value above 1.5 characterizes the cell as having a high level of aromatase expression; and
a staining intensity value below 1.5 characterizes the cell as having a low level of aromatase expression.

12. The method of claim 10, wherein a staining intensity value is quantified using image analysis software.

13. The method of claim 10, wherein the numerical scale of staining intensity values is derived from a plurality of stained cells having a plurality of levels of aromatase expression.

14. The method of claim 10, wherein a low level of aromatase expression identifies the patient as having a greater probability of surviving for at least 1, 2, 3, 4 or 5 years from the time of the examination than does a patient identified as having a high level of aromatase expression.

15. The method of claim 10, wherein a high level of aromatase expression identifies the patient as a candidate for treatment with an aromatase inhibitor.

16. The method of claim 1, wherein the level of aromatase polypeptide expressed in the non-small cell lung carcinoma is observed indirectly by:
using a polymerase chain reaction to observe the level of aromatase mRNA in the cell that encode the aromatase polypeptide; and
correlating the level of aromatase mRNA observed in the cell with the level of aromatase polypeptide expressed in the cell.

17. The method of claim 1, wherein the level of aromatase polypeptide expressed by the non-small cell lung carcinoma is observed using a high-density tissue microarray.

18. A method of obtaining information useful for determining a prognosis or therapy for a female patient having or suspected of having a human non-small cell lung carcinoma, the method comprising:
observing a level of aromatase (SEQ ID NO: 2) polypeptide expressed in a tissue sample obtained from the patient that includes or is suspected of including the human non-small cell lung carcinoma cells; and
correlating the level of aromatase polypeptide observed in the cells with a prognosis or therapy for non-small cell lung carcinoma that is associated with the observed level of aromatase (SEQ ID NO: 2) polypeptide expressed by the cells.

19. The method of claim 18, wherein the level of aromatase polypeptide expressed in the tissue sample is determined using an immunohistochemical staining technique; and
the method further comprises determining the percentage of cells in the tissue sample that are stained in the immunohistochemical staining technique.

20. The method of claim 18, wherein:
the level of aromatase polypeptide expressed in the tissue sample is determined using an immunohistochemical staining technique;
the method is preformed on a plurality of patients; and
the plurality of patients are stratified into those having a staining intensity in the immunohistochemical staining technique that is either above or below a mean or median staining intensity value on a numerical scale of staining intensity values derived from a plurality of stained cells having a plurality of levels of aromatase expression.

21. The method of claim 20, wherein the stratification is used to:
predict the probability of the patient surviving for at least 1, 2, 3, 4 or 5 years from the time of the examination; or
identify patients likely to respond to a therapeutic regimen comprising a aromatase inhibitor.

22. The method of claim 18, wherein the level of aromatase polypeptide expressed in the non-small cell lung carcinoma is observed by:
observing the level of aromatase mRNA in the cell that encode the aromatase polypeptide; and
correlating the level of aromatase mRNA observed in the cell with the level of aromatase polypeptide expressed in the cell.

23. The method of claim 22, wherein the aromatase mRNA is observed in the cell using a polynucleotide that hybridizes to the aromatase mRNA.

24. The method of claim 18, further comprising determining if a cell in the tissue sample is a stage I/II non-small cell lung carcinoma.

25. The method of claim 18, further comprising obtaining information useful for determining a prognosis or therapy for a female patient by:
determining if the patient has a lifetime history of smoking more than 100 cigarettes; or
determining the age of the patient.
